(19) United States
(12) Patent Application Publication
(10) Pub. No.: US 2015/0079631 A1
(43) Pub. Date: Mar. 19, 2015

(54) MICROVESICLE-MEDIATED DELIVERY OF THERAPEUTIC MOLECULES

(71) Applicant: THE GENERAL HOSPITAL CORPORATION, Boston, MA (US)

(72) Inventors: Xandra O. Breakefield, Newton, MA (US); Mehmet Fatih Bolukbasi, Worchester, MA (US); Arda Mizrak, San Francisco, CA (US); Okay Saydam, Vienna (AT)

(73) Assignee: THE GENERAL HOSPITAL CORPORATION, Boston, MA (US)

(21) Appl. No.: 14/384,056

(22) PCT Filed: Mar. 13, 2013
(86) PCT No.: PCT/US13/30730

§ 371 Date: Sep. 9, 2014

Related U.S. Application Data
(60) Provisional application No. 61/683,033, filed on Aug. 14, 2012.

(51) Int. Cl. C12P 21/00; A61K 9/127; C12P 19/34

(52) U.S. Cl. CPC C12P 21/00; C12P 19/34; A61K 9/127
USPC 435/69.1

(57) ABSTRACT

Disclosed herein are methods to produce extracellular vesicles such as microvesicles that contain therapeutic molecules. Such therapeutic molecules can be nucleic acid or protein or combinations thereof. Methods to deliver the therapeutic molecules to a cell are also disclosed. Therapeutic methods of treatment of disease such as cancer by delivering conditionally a lethal molecule to the cancer cells by administering microvesicles are also disclosed.

### CROSS REFERENCE TO RELATED APPLICATIONS

This application claims the benefit under 35 U.S.C. §119(e) of U.S. Provisional Application No. 61/611,837, filed Mar. 16, 2012, and U.S. Provisional Application No. 61/683,033, filed Aug. 14, 2012, the contents of each of which are incorporated herein by reference in their entirety.

### GOVERNMENTAL SUPPORT

This invention was made with Government support under grant CA141150 awarded by the National Institutes of Health, National Cancer Institute, and under grant NS037409 awarded by the National Institutes of Health, National Institute of Neurological Disorders and Stroke. The Government has certain rights in the invention.

### FIELD OF THE INVENTION

The present invention relates to the field of extracellular vesicles and therapeutics.

### BACKGROUND OF THE INVENTION

Cancer therapeutic strategies include gene delivery to target cancer cells in order to replace dysfunctional tumor suppressor genes, elicit immune rejection or drive tumor cells into apoptotic pathways. To date, several biological delivery vehicles, including DNA, cationic liposomes, viral vectors, and siRNA nanoparticles have been used with advantages and limitations.^{1,2} Naked genetic materials are inefficient in targeting because of rapid clearance by extracellular nucleases.³ Liposomes can efficiently load genetic molecules, however, their clearance rate and immunogenicity put limitations on clinical applications.⁴ Many viral gene delivery vehicles, such as Herpes Simplex Virus (HSV), adenovirus, adeno-associated virus (AAV) and retrovirus/lentivirus vectors can efficiently transfer genetic material inside tumor cells, however, with limitations in some cases, such as small packaging capacity, loss with cell division, immune response to viral particles and insertional mutagenesis.^{5,6,7} Virus and liposome delivery tools are recognized by the host immune system as foreign particles resulting in generation of antibodies against them and thereby decreasing transgene delivery dramatically upon repeated administration, as well as causing immune rejection of transduced cells.⁸ Lipid nanoparticles are susceptible to opsonin and complement system-mediated clearance in the blood and following uptake into endosomes can trigger the activation of TLR7/8 resulting in transgene silencing.⁹ Polymeric siRNA nanoparticles such as polyethylenimine (PEI)-siRNA can be subjected to rapid clearance upon binding to serum proteins. Although polymeric nanoparticles can escape from endosomes through the proton sponge effect of PEI and effectively deliver siRNA to the cytosol, leakage of endosomal and lysosomal membrane components can result in release of cathepsin B and inflammasome activation.¹⁰ Additionally, PEI complexes, as well as virus vectors, tend to accumulate in lung, liver and spleen, which make targeting to other tissues and tumors challenging.¹¹

Microvesicles (MVs), on the contrary, are a natural mammalian delivery system used by many cell types under both normal physiological and pathological conditions.¹² They include a variety of different vesicle types, variously termed exosomes, shed MVs and microparticles, extracellular vesicles, typically ranging in size from 50 to 800 nm in diameter. They are released into the extracellular environment through fusion of endosome-derived multivesicular bodies with the plasma membrane and by budding from the plasma membrane.^{13,14,15,16,12,17} These small vesicles are released by cancer cells in abundance as a means to modify the tumor micro-environment.^{18,19,20,21,22,23} Recent studies have shown that MVs can function to carry a multitude of cargos, including mRNAs, proteins, miRNA, non-coding RNAs, and DNA between cells.^{24,22,23,25,26,27}

### SUMMARY OF THE INVENTION

Aspects of the invention relate to a method of producing therapeutic microvesicles comprising the steps, isolating donor cells from a subject, genetically modifying the donor cells to express a therapeutic nucleic acid and/or protein, and isolating microvesicles (extracellular vesicles) produced by the donor cells, to thereby produce therapeutic microvesicles. In one embodiment, the cells are genetically modified by transduction with an expression vector encoding the therapeutic nucleic acid and/or protein in expressible form. In one embodiment, the therapeutic nucleic acid is selected from the group consisting of mRNA, shRNA, miRNAs, ncRNA, and aptamer. In one embodiment, the mRNA encodes a therapeutic protein. In one embodiment, the mRNA molecule encodes a protein that activates a prodrug. In one embodiment, the protein is cytosine deaminase fused in frame with uracil phosphoribosyltransferase and the prodrug is 5-fluorocytosine (5-FC). In one embodiment, the therapeutic protein is an enzyme. In one embodiment, therapeutic protein is Caspase-3, Caspase 7, Caspase-9, Caspase-8, Bax, Bid, Bad, Bak, BCL2L11, p53, PUMA, Diablo/SMAC, S-TRAIL, or combinations thereof. In one embodiment, the therapeutic protein activates a prodrug. In one embodiment, the therapeutic protein is naturally deficient in a disease. In one embodiment, the therapeutic protein is cytosine deaminase fused in frame with uracil phosphoribosyltransferase, and the prodrug is 5-FC. In one embodiment, step b) of the method results in overexpression of the therapeutic nucleic acid and/or protein in the donor cells. In one embodiment, the donor cells are epithelial cells, skin fibroblasts, mast cells, T lymphocytes, B lymphocytes or dendritic cells.

Another aspect of the invention relates to a therapeutic microvesicle preparation produced by the herein described methods.

Another aspect of the invention relates to a method of delivering a therapeutic molecule to a subject comprising, administering an effective amount of a therapeutic microvesicle preparation produced by the herein described methods to the subject. In one embodiment, administering is local or systemic. In one embodiment, local administration is by injection. In one embodiment, injection is into an organ or a tumor.

Another aspect of the invention relates to a method of delivering a therapeutic molecule to a cell comprising, contacting the cell with an effective amount of a therapeutic microvesicle preparation produced by the herein described methods. In one embodiment, the cell is in vivo. In one embodiment, the cell is within an organ or tumor. In one embodiment, the therapeutic microvesicle preparation is produced ex vivo from donor cells of a subject.

Another aspect of the invention relates to a method of treating a subject for a tumor comprising, administering a therapeutically effective amount of a therapeutic microvesicle preparation containing a therapeutic molecule selected from the group consisting of a mRNA encoding cytosine deaminase (CD) fused in-frame with uracil phosphoribosyltransferase (UPRT), cytosine deaminase (CD) fused in-frame with uracil phosphoribosyltransferase (UPRT), or a combination thereof, to the subject to thereby contact the therapeutic microvesicle preparation to a tumor in the subject, and administering a therapeutically effective amount 5-FC to the subject to thereby contact the tumor. In one embodiment, administering step a) is by injection into the tumor. In one embodiment, administering step b) is systemic. In one embodiment, the therapeutic microvesicle preparation is prepared from donor cells isolated from the subject. In one embodiment, the donor cells are selected from the group consisting of epithelial cells, skin fibroblasts, mast cells, T lymphocytes, B lymphocytes and dendritic cells.

### DEFINITIONS

“Microvesicles”, as the term is used herein, refers to membrane-derived microvesicles, which includes a range of extracellular vesicles, including exosomes, microparticles and shed microvesicles secreted by many cell types under both normal physiological and pathological conditions. The methods and compositions described herein can be applied to microvesicles of all sizes; in one embodiment, 30 to 200 nm, in one embodiment, 30 to 800 nm, in one embodiment, up to 2 um. The methods and compositions described herein can also be more broadly applied to all extracellular vesicles, a term which encompasses exosomes, shed microvesicles, oncosomes, ectosomes, and retroviral-like particles.

The term “purified” when used in reference to a microvesicle/extracellular vesicle refers to the fact that it is removed from the majority of other cellular components from which it was generated or in which it is typically present in nature. The microvesicle/extracellular vesicle of the invention are typically prepared to the state where they are purified or semi-purified.

The term “target nucleic acid molecule” or “target RNA” is used herein to refer to a nucleic acid molecule that is specifically engineered to be enriched in microvesicles produced by a cell in which it is expressed, by the methods described herein.

The term “in expressible form” when used in the context of a DNA molecule means operably linked (e.g., located within functional distance) to sequences necessary for transcription of the DNA into RNA by the RNA polymerase transcription machinery found in eukaryotic cells (e.g., promoter sequences, and other 5′ regulatory sequences). One example is a DNA molecule in the context of an expression vector. Expression can refer to transcription of DNA into RNA, and when protein coding sequences are involved, expression may also encompass translation of the mRNA into protein.

The term “operably linked” is used herein to refer to a functional relationship of one nucleic acid sequence to another nucleic acid sequence. Nucleic acid sequences are “operably linked” when placed into a functional relationship with one another. For example, a promoter or enhancer is operably linked to a coding sequence if it affects the transcription of the sequence; or a ribosome binding site is operably linked to a coding sequence if it is positioned so as to facilitate translation. The DNA sequences being linked may be contiguous, or separated by intervening sequences, and when necessary in the same reading phase and/or appropriate orientation. Linking is accomplished, for example, by ligation at convenient restriction sites. If such sites do not exist, the synthetic oligonucleotide adaptors or linkers are used in accordance with conventional practice.

As the term is used herein, “transfection” refers to the introduction of nucleic acid into a cell (e.g., for the purpose of expression of the nucleic acid by the cell). Examples of methods of transfection are electroporation, calcium phosphate, lipofection, and viral infection utilizing a viral vector. Typically nucleic acid is introduced into a cell in expressible form. That means that the nucleic acid is in the appropriate context of regulatory sequences such that the cellular machinery will recognize it and process it (e.g., transcribe RNA from DNA, translate protein from RNA). In one embodiment, a nucleic acid is in expressible form when it is inserted into an expression vector in the proper orientation to confer expression.

An “effective amount” as the term is used herein, is used to refer to an amount that is sufficient to produce at least a reproducibly detectable amount of the desired results. An effective amount will vary with the specific conditions and circumstances. Such an amount can be determined by the skilled practitioner for a given situation.

The term “therapeutically effective amount” refers to an amount that is sufficient to effect a therapeutically significant reduction in one or more symptoms of the condition when administered to a typical subject who has the condition. A therapeutically significant reduction in a symptom is, e.g. about 10%, about 20%, about 30%, about 40%, about 50%, about 60%, about 70%, about 80%, about 90%, about 100%, or more as compared to a control or non-treated subject.

The term “treat” or “treatment” refers to therapeutic treatment wherein the object is to eliminate or lessen symptoms. Beneficial or desired clinical results include, but are not limited to, elimination of symptoms, alleviation of symptoms, diminishment of extent of condition, stabilized (i.e., not worsening) state of condition, delay or slowing of progression of the condition.

Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with unwanted neuronal activity. In addition, the methods and compositions described herein can be used to treat domesticated animals and/or pets.

The terms “patient”, “subject” and “individual” are used interchangeably herein, and refer to an animal, particularly a human, to whom treatment including prophylaxic treatment is provided. This includes human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. “Mammal” refers to any animal classified as a mammal, including humans, non-human primates, domestic and farm animals, and zoo, sports, or pet animals, such as dogs, cats, cattle, horses, sheep, pigs, goats, rabbits, etc. Patient or subject includes any subset of the foregoing, e.g., all of the above, but excluding one or more groups or species such as humans, primates or rodents. A subject can be male or female. A subject can be a fully developed subject (e.g., an adult) or a subject undergoing the developmental process (e.g., a child, infant or fetus).

The terms “cell proliferative disorder” and “proliferative disorder” refer to disorders that are associated with some degree of abnormal cell proliferation. In one embodiment, the cell proliferative disorder is a tumor. In one embodiment, the cell proliferative disorder is cancer.

The term “tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein. In one embodiment, tumors are benign. Examples of benign tumors include, without limitation, schwannomas, lipoma, chondroma, adenomas (e.g., hepatic adenoma), and benign brain tumors (e.g., glioma, astrocytoma, meningioma).

The terms “cancer” and “cancerous” refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

The term “inhibiting tumor cell growth or proliferation” means decreasing a tumor cell's growth or proliferation by at least 10%, 20%, 30%, 40%, 50%, 60%, 70%, 80%, 90%, 95%, or 100%, and includes inducing cell death in a cell or cells within a cell mass.

The term “tumor progression” refers to all stages of a tumor, including tumorigenesis, tumor growth and proliferation, invasion, and metastasis.

The term “inhibiting tumor progression” means inhibiting the development, growth, proliferation, or spreading of a tumor, including without limitation the following effects inhibition of growth of cells in a tumor, (2) inhibition, to some extent, of tumor growth, including slowing down or complete growth arrest; (3) reduction in the number of tumor cells; (4) reduction in tumor size; (5) inhibition (i.e., reduction, slowing down or complete stopping) of tumor cell infiltration into adjacent peripheral organs and/or tissues; (6) inhibition (i.e. reduction, slowing down or complete stopping) of metastasis; (7) increase in the length of survival of a patient or patient population following treatment for a tumor; and/or (8) decreased mortality of a patient or patient population at a given timepoint following treatment for a tumor.

A tumor “responds” to a particular agent if tumor progression is inhibited as defined above.

### BRIEF DESCRIPTION OF THE DRAWINGS

FIG. 1 is an illustration of one example of engineering of MVs as novel gene delivery tools. The therapeutic expression vector, in this case CD-UPRT-EGFP is delivered into donor cells via DNA transfection or infection with a viral vector. A few days later, MVs enriched with expressed mRNAs/protein are harvested from the conditioned medium and concentrated by ultracentrifugation. Recipient cancer cells/tumor are treated with those MVs followed a few days later by an administration of an activating agent, in this case the prodrug, 5-FC. CD and UPRT converts 5-FC to 5-FdUMP, an irreversible inhibitor of thymidine synthetase, thereby restricting the production of dTMP. Depletion of dTTP results in inhibition of DNA synthesis and leads to apoptosis of cancer cells.

FIG. 2a-FIG. 2d is a collection of photographs of experimental results that show the CD-UPRT-EGFP mRNA and protein are enriched in MVs. (FIG. 2a) Total RNA was isolated from MVs collected from HEK-293T cells transfected with pCD-UPRT-EGFP vector and qRT-PCR was performed for the CD-UPRT and GAPDH mRNAs. The end PCR products were loaded onto agarose gel. CD-UPRT mRNAs levels to GAPDH are shown. (FIG. 2b) After MV collection in (a), DNase and RNase treatment were performed on MVs and/or their contents, as indicated. Similar qRT-PCR reactions were performed as in (a) and the end product DNA was loaded onto agarose gels. A representative agarose gel from three independent qRT-PCRs is shown. (FIG. 2c) Nucleic acid content of MVs were treated with DNase or left non treated and then qPCRs were performed directly without RT and PCR products were loaded onto agarose gels (FIG. 2d) Western blot analysis was carried out on MVs (40 μg protein) collected from HEK-293T transfected with pEGFP or pCD-UPRT-EGFP or non-untransfected. HEK-293T cell lysates (10 μg) from pCD-UPRT-EGFP plasmid transfected cells were used as a positive control for the CD-UPRT-EGFP fusion protein.

FIG. 3a-FIG. 3b show photographic and graphic representations of experimental results which indicate MVs carrying the CD-UPRT-EGFP mRNA/protein are functional in recipient cells. (FIG. 3a) Three days after treatment of HEI-193 with MVs carrying CD-UPRT-EGFP mRNA/protein in increasing concentrations (1, 3, and 5 μL), total RNA was isolated and RT-PCR was performed for the CD-UPRT-EGFP and GAPDH mRNAs and products resolved by ethidium bromide gel electrophoresis. (FIG. 3b) MTT assays were performed on HEI-193 cells 3 days after exposure to MVs containing CD-UPRT-EGFP mRNA/protein or control EGFP, in both cases with prodrug 5-FC treatment. The experiments were performed in triplicate, and the values are expressed as the mean±SD (***, p<0.001, Student's t-test).

FIG. 4a-FIG. 4b show photographic and graphic representation of experimental results which indicate intratumoral delivery of MVs carrying the CD-UPRT-EGFP mRNA/protein inhibited schwannoma tumor growth in vivo. (FIG. 4a) HEI-193FC cells were injected (3×10⁴ cells in 1 μl of culture medium) into the sciatic nerve of nude mice starting 3 weeks after tumor implantation. MVs harvested from HEK-293T cells transfected with either pCD-UPRT-GFP or pEGFP plasmids were injected weekly into tumors for 2 months (using 1 μl per tumor out of 20 μl MVs isolated from 4×10⁷ cells) and prodrug 5-FC was given daily through intraperitoneal injections following the initial MV injection. Tumor growth was monitored by in vivo bioluminescence imaging using the Xenogen IVIS system to monitor photon emission. Bioluminescent images are shown with a pseudocolor bar to indicate degree of bioluminescence at 1, 28, and 56 days after tumor cell implantation. (FIG. 4b) The average photon counts of the groups is represented as an index of tumor growth, starting with 100% as the initial value. The values are expressed as the mean±SD (***, p<0.001, Student's t-test).

FIG. 5 is a bar graph of experimental results that indicate the effect of MV-CD-UPRT-EGFP and MV-EGFP on cell viability after purification by sucrose gradient ultracentrifugation. HEK-293T cells were transfected either with pCD-UPRT-EGFP or pEGFP and 2 days later MVs were purified by sucrose gradient ultracentrifugation. HEI-193 cells were treated with these MVs for 3 days followed by MTT assays. The experiments were performed in triplicate, and the values are expressed as the mean±SD (**, p<0.01, Student's t-test).

FIG. 6a-FIG. 6b are graphs of experimental results that represent MVs size comparison by NanoSight. MVs were purified either by ultracentrifugation (FIG. 6a) or sucrose gradient ultracentrifugation (FIG. 6b) and characterized by NanoSight.

FIG. 7a-FIG. 7b are a collection of photographs showing representative experimental results that indicate MVs uptake of HEI-193 cells after purification by sucrose gradient ultracentrifugation. HEI-193 cells were treated with MVs isolated by sucrose gradient ultracentrifugation for 16 hours and 24 hours. Cells were fixed after treatment period and examined using a fluorescence microscope for EGFP, using DAPI to stain nuclei. Magnification=40×.

FIG. 8a-FIG. 8c are graphic and photographic representations of experimental results that indicate the genetically engineered MVs kill other cancer cell types. MTT cell viability assay was performed after exposure to MVs, as in FIG. 3b, on glioma and meningioma cell lines, U87 (FIG. 8a) and SF4433 (FIG. 8b), respectively, with subsequent treatment with 5-FC. (FIG. 8c) RT-PCR reactions were performed as in FIG. 3a from total RNAs isolated from U87 and SF4433 cells for CD-UPRT and GAPDH mRNAs. Non-treated U87 cells were used as a negative control.

FIG. 9a-FIG. 9b are photographs showing representative experimental results that indicate CD-UPRT-EGFP expression increases in recipient cells over time. HEI-193 cells were treated for 16, and 24 hours with MVs collected from HEK-293T cells transfected with pCD-UPRT-EGFP vector (FIG. 9a) or pEGFP (FIG. 9b). Cells were fixed after treatment period and examined using a fluorescence microscope for EGFP, using DAPI to stain nuclei. Magnification=40×.

FIG. 10a-FIG. 10b are photographs showing representative experimental results that indicate D-UPRT-EGFP and pEGFP expression patterns in plasmid DNA transfected HEK-293T cells. HEK-293T cells were transfected either with pCD-UPRT-EGFP expression plasmid (FIG. 10a) or pEGFP plasmid (FIG. 10b) and then fixed at indicated time points. Cells were examined using a fluorescence microscope for EGFP, using DAPI to stain nuclei. Magnification=40×. These experiments were performed in triplicate and the values are expressed as the mean±SD (***, p<0.001, Student's t-test).

FIG. 11a-FIG. 11b show photographic and graphic representations of experimental results which indicate inhibition of schwannoma tumor growth after intratumoral delivery of MVs carrying the CD-UPRT-EGFP mRNA/protein. Similar in vivo experiments are performed as in FIG. 4 and tumor growth was monitored by in vivo bioluminescence imaging using a CCD camera. (FIG. 11a) Bioluminescent images are shown with a pseudocolor bar to indicate degree of bioluminescence at 1, 28, and 56 days after tumor cell implantation. (FIG. 11b). The average photon counts of the groups is represented as an index of tumor growth, starting with 100% as the initial value. The values are expressed as the mean±SD (***, p<0.001, Student's t-test).

FIG. 12a-FIG. 12b are graphs of experimental results that indicate a correlation between MVs numbers and RNA content. HEK-293T cells were transfected either with pCD-UPRT-EGFP or pEGFP vectors and MVs were isolated from these transfected cells, as described in Material and Methods. (FIG. 12a) MVs were counted using a NaonoSight, NS500. (FIG. 12b) RNA was isolated from same MVs and measured by NanoDrop. Fold increases in MVs numbers and RNA amount were calculated accordingly to measurements of MVs isolated from 1 to 15 ml of MV medium.

### DETAILED DESCRIPTION OF THE INVENTION

Microvesicles (MVs) play an important role in intercellular communication by carrying mRNAs, microRNAs (miRNAs), non-coding RNAs, proteins and DNA from cell to cell. Disclosed herein is evidence of successful delivery of therapeutic molecules (mRNA/protein) via MVs for treatment of cancer. MV were generated by genetic engineering through expression of high levels of the suicide gene mRNA and protein—cytosine deaminase (CD) fused to uracil phosphoribosyltransferase (UPRT) in MV donor cells. MVs were isolated from these cells and used to treat pre-established nerve sheath tumors (schwannomas) in an orthotopic mouse model. The MV-mediated delivery of CD-UPRT mRNA/protein by direct injection into schwannomas led to regression of these tumors upon systemic treatment with the prodrug (5-fluorocytosine), which is converted within tumor cells to 5-fluorouracil—an anticancer agent. Taken together, these studies indicate that MVs, and other extracellular vesicles, can be generated to contain molecules typically exogenous to a cell, and also can serve as cell-derived “liposomes” to effectively deliver therapeutic molecules (e.g., nucleic acids and proteins) in the treatment of diseases.

Aspects of the invention relate to the use of microvesicles carrying therapeutic molecules and methods of generating such microvesicles. Microvesicles which contain a therapeutic molecule are referred to herein as “therapeutic microvesicles”. One aspect of the invention relates to a method of producing therapeutic microvesicles from a population of cells. The experiments reported herein indicate that the content of the microvesicles produced by a cell can be altered by scientific manipulation of the cell (donor cells) to shift the contents of molecules within the cell. Although the microvesicles generated will also contain other molecules derived from the donor cells (e.g, naturally present within the microvesicles), the molecules contained within the microvesicles can be enriched for a given molecule by the methods described herein. Manipulation of a cell or population of cells to increase the content of a therapeutic molecule into the microvesicles produced therefrom is referred to herein as “enriching” the microvesicles for the therapeutic molecule. Such microvesicles produced therefrom are referred to as “enriched” for the therapeutic microvesicle. The microvesicles generated by the cells are typically isolated in the form of a population, based on similar size or density, otherwise referred to as a “microvesicle preparation”.

The therapeutic microvesicles for use in the methods described herein will be enriched for the therapeutic molecules by cellular manipulation such as engineering expression of an exogenous nucleic acid into the cell. In one embodiment, the method comprises genetically modifying cells in vitro to adequately express one or more therapeutic molecules (e.g., a nucleic acid and/or protein) such that they are incorporated in sufficient amounts into microvesicles produced by the cells. Microvesicles are then isolated from those cells (herein referred to as donor cells). The microvesicles generated by the cells can be isolated in the form of a population, typically based on similar size or density, otherwise referred to as a “microvesicle preparation”. The microvesicles of the preparation can be analyzed to determine the level of content of the therapeutic molecules therein by standard methods. The method of preparation can involve further steps of isolation or fractionation of the microvesicles (e.g., to further purify the microvesicles from other preparation components, or to select for a subset of the microvesicles obtained). Such selection can be based on size or content.

The cells which are modified to become donor cells may or may not already contain some level of the therapeutic molecule(s). In one embodiment, the therapeutic molecule is not naturally present in the donor cells, but is exogenous to the cells. Alternatively, the therapeutic molecule may be naturally present and expressed in the donor cells with the natural expression increased or enhanced by manipulation of the cells (e.g., introduction of a regulatory factor that increases the natural expression or introduction of an exogenous nucleic acid encoding the therapeutic molecule to thereby increase expression by the cells). Such enrichment of the therapeutic molecules into microvesicles produced by the donor cells (the number of microvesicles containing the molecules and/or the amount of the molecules in relation to other molecules in the microvesicle) for the therapeutic molecule can be achieved by a variety of methods. The specific method will depend upon the specific molecules and the specific cell type used.

One such method of enriching microvesicle content for a therapeutic molecule is to genetically modify the cells to express the encoding nucleic acid at a relatively high level. This can be accomplished by transfecting the cells with an expression vector comprising a nucleic acid that encodes the therapeutic molecule(s). The recipient cells may express the transfected nucleic acid transiently, or may incorporate the transfected nucleic acid into their genome and express the nucleic acid stably. High levels of expression can be achieved by utilizing a strong promoter. High levels of expression may also be achieved by selection and propagation of fairly high expressing cell recipients from the transfected population of cells. Levels of expression of specific molecules can be determined through standard means in the art and can be quantitated by comparison to expression levels of endogenous molecules such as housekeeping genes or proteins.

Another method of enriching microvesicle content for the therapeutic molecule is through the manipulation of naturally occurring microvesicle targeting methods (e.g., the incorporation of zip code like sequences into the nucleic acid in the genetic modification process). In one embodiment, specific microvesicle targeting sequences are linked to nucleic acids encoding the therapeutic molecules, and transfected into the donor cells in expressible form. Such targeting methods are described in PCT/US13/21879, the contents of which are incorporated herein by reference.

Another method of enriching microvesicle content for the therapeutic molecule is through incorporation of a selectable moiety that co-localizes with the therapeutic molecules. Microvesicles containing the selectable moiety can be enriched or selected away from non-selected microvesicles in a preparation by virtue of the moiety's properties, to thereby generate an enriched preparation. Examples of useful selectable moieties include, without limitation, binding moieties and visually detectable moieties such as fluorescent molecules.

The incorporation of more than one therapeutic molecule into the microvesicles of the preparation is also envisioned. For example two or more mRNAs (e.g., having different nucleotide sequences) can be incorporated, two or more proteins (having different amino acid sequences) can be incorporated, and combinations of mRNA, protein, and possibly other forms of therapeutic molecules (e.g., siRNA) can also be incorporated into the microvesicles of the preparation. The combining of two or more independently generated microvesicle preparations which each have unique complements of therapeutic molecules, to thereby produce one therapeutic microvesicle preparation is also envisioned.

These and other various methods for enrichment can be used alone or in combination with one another to produce a microvesicle preparation containing an effective amount of the therapeutic molecule(s).

Microvesicle preparations containing an effective amount of the desired therapeutic molecules can then be used to deliver their content to target cells (e.g., in vivo, or in vitro). Alternatively, the microvesicles within the preparation can be modified, or stored for future use. In one embodiment, the method of preparation of the therapeutic microvesicles is ex vivo, in that donor cells are isolated from a subject into whom the microvesicles will be therapeutically introduced.

### Microvesicle Preparation

One aspect of the invention relates to a microvesicle or a preparation thereof, that contains one or more therapeutic molecules described herein. Such a microvesicle or preparation is produced by the herein described methods. As the term is used herein, a microvesicle preparation refers to a population of microvesicles obtained/prepared from the same cellular source. Such a preparation is generated, for example, in vitro, by culturing cells expressing the nucleic acid molecule of the instant invention and isolating microvesicles produced by the cells. Methods of isolating such microvesicles are known in the art (Thery et al., Isolation and characterization of exosomes from cell culture supernatants and biological fluids, in Current Protocols Cell Biology, Chapter 3, 322, (John Wiley, 2006); Palmisano et al., (Mol Cell Proteomics. 2012 August; 11(8):230-43) and Waldenström et al., ((2012) PLoS ONE 7(4): e34653.doi: 10.1371/journal.pone.0034653)), some examples of which are described herein. Such techniques for isolating microvesicles from cells in culture include, without limitation, sucrose gradient purification/separation and differential centrifugation, and can be adapted for use in the invention.

In one embodiment, the microvesicles are isolated by gentle centrifugation (e.g., at about 300 g) of the culture medium of the donor cells for a period of time adequate to separate cells from the medium (e.g., about 15 minutes). This leaves the microvesicles in the supernatant, to thereby yield the microvesicle preparation. In one embodiment, the culture medium or the supernatant from the gentle centrifugation, is more strongly centrifuged (e.g., at about 16,000 g) for a period of time adequate to precipitate cellular debri (e.g., about 30 minutes). This leaves the microvesicles in the supernatant, to thereby yield the microvesicle preparation. In one embodiment, the culture medium, the gentle centrifuged preparation, or the strongly centrifuged preparation is subjected to filtration (e.g., through a 0.22 um filter or a 0.8 um filter, whereby the microvesicles pass through the filter. In one embodiment, the filtrate is subjected to a final ultracentrifugation (e.g. at about 110,000 g) for a period of time that will adequately precipitate the microvesicles (e.g. for about 80 minutes). The resulting pellet contains the microvesicles and can be resuspended in a volume of buffer that yields a useful concentration for further use, to thereby yield the microvesicle preparation. In one embodiment, the microvesicle preparation is produced by sucrose density gradient purification. In one embodiment, the microvesicles are further treated with DNAse (e.g., DNAse I) and/or RNAse and/or proteinase to eliminate any contaminating DNA, RNA, or protein, respectively, from the exterior. In one embodiment, the microvesicle preparation contains one or more RNAse inhibitors.

The molecules contained within the microvesicle preparation will comprise the therapeutic molecule. Typically the microvesicles in a preparation will be a heterogeneous population, and each microvesicle will contain a complement of molecule that may or may not differ from that of other microvesicles in the preparation. The content of the therapeutic molecules in a microvesicle preparation can be expressed either quantitatively or qualitatively. One such method is to express the content as the percentage of total molecules within the microvesicle preparation. By way of example, if the therapeutic molecule is an mRNA, the content can be expressed as the percentage of total RNA content, or alternatively as the percentage of total mRNA content, of the microvesicle preparation. Similarly, if the therapeutic molecule is a protein, the content can be expressed as the percentage of total protein within the microvesicles. In one embodiment, therapeutic microvesicles, or a preparation thereof, produced by the method described herein contain a detectable, statistically significantly increased amount of the therapeutic molecule as compared to microvesicles obtained from control cells (cells obtained from the same source which have not undergone scientific manipulation to increase expression of the therapeutic molecule). In one embodiment, the therapeutic molecule is present in an amount that is at least about 10%, 20%, 30% 40%, 50%, 60%, 70% 80% or 90%, more than in microvesicles obtained from control cells. Higher levels of enrichment may also be achieved. In one embodiment, the therapeutic molecule is present in the microvesicle or preparation thereof, at least 2 fold more than control cell microvesicles. Higher fold enrichment may also be obtained (e.g., 3, 4, 5, 6, 7, 8, 9 or 10 fold).

In one embodiment, a relatively high percentage of the microvesicle content is the therapeutic molecule (e.g., achieved through overexpression or specific targeting of the molecule to microvesicles). In one embodiment, the microvesicle content of the therapeutic molecule is at least about 10%, 20%, 30% 40%, 50%, 60%, 70% 80% or 90%, of the total (like) molecule content (e.g., the therapeutic molecule is an mRNA and is about 10% of the total mRNA content of the microvesicle). Higher levels of enrichment may also be achieved. In one embodiment, the therapeutic molecule is present in the microvesicle or preparation thereof, at least 2 fold more than all other such (like) molecules. Higher fold enrichment may also be obtained (e.g., 3, 4, 5, 6, 7, 8, 9 or 10 fold).

### Therapeutic Molecules—Nucleic Acids

A variety of different types of molecules can potentially serve as a therapeutic molecule. In one embodiment, the therapeutic molecule in the microvesicle preparation is a nucleic acid molecule. A nucleic acid molecule, as used herein, can be RNA or DNA, and can be single or double stranded. Such nucleic acid molecules include, for example, but are not limited to, nucleic acid molecules encoding proteins. Other such nucleic acid molecules, for example, may act as transcriptional repressors, antisense molecules, ribozymes, small inhibitory nucleic acid sequences (e.g., RNAi, shRNAi, siRNA, stRNA, micro RNAi (mRNAi), antisense oligonucleotides, and RNA aptamers,).

Various forms of RNA molecules are envisioned for use as a therapeutic molecule including mRNA and ncRNA (non-coding RNA). mRNA (messenger RNA) is a molecule of RNA that encodes a chemical “blueprint” for a protein product. mRNA is transcribed from a DNA template, and carries coding information to the sites of protein synthesis: the ribosomes, where the nucleic acid polymer is translated into a polymer of amino acids. mRNA comprises contiguous sequence of nucleotides arranged into codons consisting of three bases each, with each codon encoding for a specific amino acid, the stretch of contiguous in-frame codons terminating with a stop codon, which terminate protein synthesis. mRNA also contain untranslated regions (5′ and 3′ UTR). An mRNA typically will contain a polyA at the 3′ end. Many non-coding RNAs also contain a polyA site.

A non-coding RNA (ncRNA) is a functional RNA molecule that is not translated into a protein. Less-frequently used synonyms are non-protein-coding RNA (npcRNA), non-messenger RNA (nmRNA) and functional RNA (fRNA). The DNA sequence from which a non-coding RNA is transcribed is sometimes referred to as an RNA gene. Non-coding RNA include highly abundant and functionally important RNAs such as transfer RNA (tRNA) and ribosomal RNA (rRNA), as well as RNAs such as snoRNAs, microRNAs, siRNAs and piRNAs and the long non coding RNAs. Long non-coding RNAs (long ncRNAs, lncRNA) are non-protein coding transcripts longer than 200 nucleotides.

The term “short interfering RNA” (siRNA), also referred to herein as “small interfering RNA” is defined as an agent which functions to inhibit expression of a target gene, e.g., by RNAi. As used herein an “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow. shRNAs function as RNAi and/or siRNA species but differ in that shRNA species are double stranded hairpin-like structure for increased stability.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 116:281-297), comprises a dsRNA molecule.

The terms “microRNA” or “miRNA” are used interchangeably herein to refer to endogenous RNAs, some of which are known to regulate the expression of protein-coding genes at the posttranscriptional level. Endogenous microRNA are small RNAs naturally present in the genome which are capable of modulating the productive utilization of mRNA. Also included in the invention are artificial microRNAs. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule.

Aptamers are a special class of nucleic acid molecules currently in clinical trials for therapy. Aptamers are short synthetic single-stranded oligonucleotides that specifically bind to various molecular targets such as small molecules, proteins, nucleic acids, and even cells and tissues. RNA Aptamers are single-stranded RNA oligonucleotides 15 to 60 base in length that bind with high affinity to specific molecular targets; most aptamers to proteins bind with Kds (equilibrium constant) in the range of 1 pM to 1 nM similar to monoclonal antibodies. These nucleic acid ligands bind to nucleic acid, proteins, small organic compounds, and even entire organisms. Aptamers have many potential uses in intracellular processes studies, medicine and technology. Aptamers are generally selected from a biopanning method known as SELEX (Systematic Evolution of Ligands by Exponential enrichment) (Ellington et al. Nature. 1990; 346(6287):818-822; Tuerk et al., Science. 1990; 249(4968):505-510; (Ni et al., Curr Med Chem. 2011; 18(27):4206-14).

These small RNA molecules can form secondary and tertiary structures capable of specifically binding proteins or other cellular targets, and are essentially a chemical equivalent of antibodies. Aptamers are highly specific, relatively small in size, and non-immunogenic. Aptamers are presently seen as clinically relevant for diseases like cancer, HIV, and macular degeneration. Many aptamers have been clinically developed as inhibitors for targets such as vascular endothelial growth factor (VEGF) and thrombin. An aptamer based therapeutic was FDA approved in 2004 for the treatment of age-related macular degeneration and several other aptamers are currently being evaluated in clinical trials. With advances in targeted-therapy, imaging, and nanotechnology, aptamers are readily considered as potential targeting ligands because of their chemical synthesis and ease of modification for conjugation. Preclinical studies using aptamer-siRNA chimeras and aptamer targeted nanoparticle therapeutics have been very successful in mouse models of cancer and HIV (Ni et al., Curr Med Chem. 2011; 18(27):4206-14).

### Therapeutic Molecules—Proteins

In one embodiment, the therapeutic molecule is a protein. Proteins which have enzymatic and regulatory activities can be used. In one embodiment, the protein is an activator (e.g., a transcription factor) or epigenetic modifier. In one embodiment, the protein is an enzyme or a pro-enzyme. In one embodiment, the protein is an inhibitory protein.

### Expression Vectors

The donor cells are modified to express the therapeutic molecule by genetically modifying them to contain a nucleic acid molecule, in expressible form, that encodes the therapeutic molecule. This nucleic acid molecule can be delivered to the donor cells in the context of an expression vector. Such vectors are typically specifically designed for the host cell in which they are to be used (e.g., prokaryotic, eukaryotic or both).

Expression vector may be, for example, plasmid or virus vectors, and typically contain an origin of replication, a promoter and a regulator of the promoter. The recombinant expression vector may then be used to transform or transfect suitable host cells such as bacterial cells, e.g. E. coli cells, or eukaryotic cells such as yeast, insect or preferably, mammalian cells, to provide for expression of a nucleic acid sequence described herein. Suitable bacterial and eukaryotic expression vectors are commercially available and well known in the art and their use is described, e.g., in Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994).

Many mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of nucleic acids in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press: 1989) Chapters 16 and 17.

Many such vectors useful for transferring exogenous genes into target mammalian cells are available. The vectors may be episomal, e.g. plasmids, virus derived vectors such cytomegalovirus, adenovirus, etc., or may be integrated into the target cell genome, through homologous recombination or random integration, e.g. retrovirus derived vectors such MMLV, HIV-1, ALV, etc. Many viral vectors or virus-associated vectors are known in the art. Such vectors can be used as carriers of a nucleic acid construct into the cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cells genome. The constructs may include viral sequences for transfection, if desired. Alternatively, the construct may be incorporated into vectors capable of episomal replication, e.g. EPV and EBV vectors.

Expression and cloning vectors usually contain one or more regulatory sequences (e.g., a promoter) operably linked to the encoding nucleic acid sequence to direct RNA synthesis. Promoters recognized by a variety of potential host cells are well known. Promoters suitable for use with prokaryotic hosts include the beta-lactamase and lactose promoter systems (Chang et al., Nature, 275:615 (1978); Goeddel et al., Nature, 281:544 (1979)), alkaline phosphatase, a tryptophan (trp) promoter system (Goeddel, Nucleic Acids Res., 8:4057 (1980); EP 36,776), and hybrid promoters such as the tac promoter (deBoer et al., Proc. Natl. Acad. Sci. USA, 80:21-25 (1983)). Promoters for use in bacterial systems also will contain a Shine-Dalgarno (S.D.) sequence operably linked to the encoding DNA. Promoters for vectors in mammalian host cells can be obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems. The promoter sequence may be a “tissue-specific promoter,” which means a nucleic acid sequence that serves as a promoter, i.e., regulates expression of a selected nucleic acid sequence operably linked to the promoter, and which affects expression of the selected nucleic acid sequence in specific cells.

Transcription of a DNA by higher eukaryotes may be increased by inserting an enhancer sequence into the vector. Enhancers are cis-acting elements of DNA, usually about from 10 to 300 bp that act on a promoter to increase its transcription. Many enhancer sequences are now known from mammalian genes (globin, elastase, albumin, .alpha.-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be present at a position 5′ or 3′ to the coding sequence, but is preferably located at a site 5′ from the promoter.

Expression vectors used in eukaryotic host cells (yeast, fungi, insect, plant, animal, human, or nucleated cells from other multicellular organisms) may also contain sequences necessary for the termination of transcription and for stabilizing the RNA. Such sequences are commonly present at the 5′ and, occasionally 3′, untranslated regions of eukaryotic or viral DNAs or cDNAs. These regions contain nucleotide segments transcribed as polyadenylated fragments in the untranslated portion of the RNA.

### Donor Cells

Cells used to generate the therapeutic microvesicles are referred to herein as donor cells. Any cell which is capable of producing microvesicles can serve as a donor cell. Donor cells may be in vitro (e.g., a cell in culture), ex vivo, or in vivo. The cells are eukaryotic, for example mammalian cells, insect cells, invertebrate cells, and avian cells. Mammalian cells may be cells typically used in the laboratory such as mouse, rat, human, and non-human primate cells. A variety of different cells types are envisioned for use in the invention. In one embodiment, the cells are mammalian, and comprise the nucleic acid molecule encoding the therapeutic molecule in expressible form. In one embodiment, the cells express the nucleic acid molecule that encodes the therapeutic molecule. Although the cells may express the therapeutic molecule naturally at low levels, the instant invention involves genetically engineering the cells to express the therapeutic molecule at higher than natural levels. In that respect, the therapeutic molecule expressed is referred to as exogenous to the donor cells. In one embodiment, the donor cells result from transient transfection of an expression vector comprising the nucleic acid molecule. In one embodiment, the donor cells result from stable transfection of the nucleic acid molecule described herein via an expression vector.

Microvesicles are secreted naturally by various types of cells, any of which may be used as donors cells in the herein described methods. Cells particularly suited for microvesicle secretion are epithelial cells, tumor cells and certain cells of the immune system (mast cells, T and B lymphocytes, dendritic cells, especially Langerhans cells). In one embodiment, the cell is a eukaryotic cell comprising internal vesicles for secretion. In one embodiment the cell is of the type which can be cultured easily. In one embodiment the cell is capable of exocytosis. In one embodiment, the cell is of a type which is readily genetically modifiable. In one embodiment, the cell secretes the internal vesicles when exposed to an external stimulus. Cells that can produce microvesicles include, without limitation, skin fibroblasts, mast cells, T and B lymphocytes and dendritic cells (for example Langerhans cells), or cells derived from these cell types, and cells or cell lines modified by genetic engineering so as to render them capable of secreting microvesicles.

In one embodiment, the cell is a continuous cell line or a tumor-derived cell line (e.g., originating from the subject to whom the generated microvesicles will be administered). In one embodiment, the cell type is useful to generate microvesicles in vitro. In one embodiment the cell is a primary cell, obtained from a multicellular organism and grown or propagated in the laboratory for a short period of time (e.g., 10 or fewer passages, 50 or fewer passages, 100 or fewer passages). Such a primary cell may be a cell obtained from a subject, to which microvesicles produced therefrom, or the cells engineered to produce therapeutic microvesicles, will be administered. In one embodiment, the cells are immature dendritic cells (e.g., generated from harvested bone marrow). In one embodiment, the immature dendritic cells are devoid of T-cell activators (e.g., MHC-II and/or CD86). Methods of producing microvesicles from immature dendritic cells are known in the art and can be adapted for use with the instant invention (US Published Patent Application 2004/0241176).

### Donor Cell Modifications

The cells in culture are genetically engineered to express the therapeutic molecule by transfection (stable or transient) with an expression vector. The method involves generating an expression vector comprising the DNA molecule that encodes the therapeutic molecule (such as an RNA molecule or a protein) in expressible form. Transfection is performed under conditions suitable for expression of the DNA molecule. Various DNA molecules are envisioned for use in this invention. Typically the DNA molecule will comprise regulatory sequences necessary and sufficient for transcription into an RNA molecule (e.g., one of the various forms discussed herein) by cellular machinery. In one embodiment, the DNA molecule encodes a protein and is first transcribed into an mRNA. The DNA that encoded the mRNA may contain splice sites, requiring that the mRNA be processed, or may alternatively lack splice sites (e.g., a cDNA). In another embodiment, the DNA molecule serves as a regulatory molecule. For example, the DNA molecule may encode for an siRNA.

Donor cells which express the therapeutic molecule(s) (e.g., mRNA and/or protein) are subjected to conditions suitable for production of microvesicles. Microvesicles generated by the cells are isolated from the culture, to thereby produce a preparation of microvesicles enriched for the specific therapeutic molecules. Conditions suitable for expression of the DNA molecule include, without limitation, conditions whereby the cells are dividing in culture. These conditions assume a minimum amount of time necessary for the uptake and expression of the DNA molecule by the transfected cells (typically 1-2 days).

The donor cells may also be further modified by genetic engineering, such as to facilitate microvesicle production, reduce microvesicle immunogenicity, or direct the microvesicles to target cells.

The donor cells may be modified to reduce immunogenicity of the microvesicles in the recipient subject. In one embodiment, the donor cells are engineered to produce immunosuppressive ligands which will be present on the resulting microvesicles. In one embodiment the donor cells are engineered to produce immunosuppressive cytokines which will be contained in the resulting microvesicles. In one embodiment, the donor cells are (e.g., a dendritic cell) engineered to express FasL³⁶.

In one embodiment, the donor cells are modified such that the microvesicles generated therefrom further comprise a targeting ligand. Such a targeting ligand may be used to direct the microvesicles to specific cells with which they will ultimately fuse. Such a targeting ligand can be produced, for example, by engineering the donor cells used to produce the microvesicles to express a protein abundantly present in exosomal membranes (e.g., Lamp2b) as a fusion protein with the targeting ligand (e.g. a neuron-specific RVG peptide). The targeting ligand can be a member of a specific binding pair, the other of which is found on the target cells (Alvarez-Erviti et al., Nature Biotechnology 29: 341-345 (2011)). In one embodiment, the targeting ligand is an antibody or antigen binding fragment thereof (e.g., a single chain antibody (scFV)) that specifically binds a marker present on a cellular target. In one embodiment, the targeting moiety facilitates crossing of the blood-brain barrier³⁸.

### Delivery of Therapeutic Molecules

The therapeutic microvesicles described herein can be used to deliver their therapeutic molecules to a target cell or population of cells. Delivery is accomplished from contacting of the microvesicles to the target cells. The target cells may be in vitro or in vivo. In vivo delivery is accomplished by administration of the therapeutic microvesicles to a subject by a route that results in contacting of the microvesicles to the target cells. For therapeutic purposes, a therapeutically effective amount is administered such that an effective amount of the therapeutic molecule is delivered to the target cells.

In one embodiment, the target cells are also subjected to a second event. A second event is used, for example, to activate a conditionally therapeutic molecule. Such a second event can be exposure to a second molecule (e.g., extracellularly or delivery intracellularly) such as a prodrug. In one embodiment the second molecule is adminstered to a subject locally to the relative location of the target cells within the subject (e.g., injection into a tumor or organ). The second molecule may be a pro-drug or an activator of the therapeutic molecule, or a molecule involved in specific delivery of the therapeutic molecule to the target cell.

The therapeutic microvesicles can be used to treat diseases which will be ameliored by the delivery or the actions of the therapeutic molecules on the target cells. In one embodiment, the disease involves or is caused by a genetic deficiency in the target cells. The molecule for which they are deficient (or encoding the molecule for which they are deficient) can be delivered to the appropriate cells via the therapeutic microvesicles. Proliferative disorders are also amenable to treatment with therapeutic microvesicles when used to deliver a molecule which inhibits cellular proliferation or induces apoptosis.

In one embodiment, the disease is a proliferative disorder, such as a tumor and the therapeutic microvesicles delivery a molecule which inhibits cell growth or promotes cell death or apoptosis. Such a molecule may be a growth regulatory molecule which has been lost in tumorigenesis. Examples of such molecules that can be delivered include, without limitation proteins from the caspase family such as Caspase-9 (P55211(CASP9_HUMAN); HGNC: 15111; Entrez Gene: 8422; Ensembl: ENSG000001329067; OMIM: 6022345; UniProtKB: P552113), Caspase-8 (Q14790 (CASP8_HUMAN); 9606 [NCBI]), Caspase-7 (P55210 (CASP7_HUMAN); 9606 [NCBI]), and Caspase-3 (HCGN: 1504; Ensembl:ENSG00000164305; HPRD:02799; MIM:600636; Vega:OTTHUMG00000133681), pro-apoptotic proteins such as Bax (HGNC: 9591; Entrez Gene: 5812; Ensembl: ENSG000000870887; OMIM: 6000405; UniProtKB: Q078123), Bid (HGNC: 10501; Entrez Gene: 6372; Ensembl: ENSG000000154757; OMIM: 6019975; UniProtKB: P559573), Bad (HGNC: 9361; Entrez Gene: 5722; Ensembl: ENSG000000023307; OMIM: 6031675; UniProtKB: Q92934), Bak (HGNC: 9491; Entrez Gene: 5782; Ensembl: ENSG000000301107; OMIM: 6005165; UniProtKB: Q166113), BCL2L11 (HGNC: 9941; Entrez Gene: 100182; Ensembl: ENSG000001530947; OMIM: 6038275; UniProtKB: O435213), p53 (HGNC: 119981; Entrez Gene: 71572; Ensembl: ENSG000001415107; OMIM: 1911705; UniProtKB: P046373), PUMA (HGNC: 178681; Entrez Gene: 271132; Ensembl: ENSG000001053277; OMIM: 6058545; UniProtKB: Q96PG83; UniProtKB: Q9BXH13), Diablo/SMAC (HGNC: 215281; Entrez Gene: 566162; Ensembl: ENSG000001840477; OMIM: 6052195; UniProtKB: Q9NR283), or a membrane bound form of TRAIL (S-TRAIL) (Humphreys et al., Adv Exp Med Biol. 2008; 615:127-58; Zhang et al., Cancer Gene Ther. 2010 May; 17(5): 334-343).

Another molecule which can be incorporated into microvesicles and delivered to target cells is a conditionally therapeutic molecule. A conditionally therapeutic molecule is a molecule which requires a second event for delivery of the therapeutic effects. Such a second event, may be, for example, the action of a second molecule in the recipient cell. Such a second molecule can be an activator, or in turn may be activated by the conditionally therapeutic molecule. One such example is a prodrug activating enzyme or a nucleic acid encoding the prodrug activating enzyme. In one embodiment, the conditionally therapeutic molecule is conditionally toxic to the recipient cell (e.g., a tumor cell). One such example is an engineered suicide gene (e.g, one encoding cytosine deaminase (CD) fused in-frame with uracil phosphoribosyltransferase (UPRT) or the encoded protein of the gene.

In one embodiment, the proliferative disorder is a tumor and the method of the invention relates to a method for inhibiting tumor progression. In such a method the therapeutic molecule can have growth inhibitory or cell killing activity. An effective amount of therapeutic microvesicle (comprising one or more therapeutic molecules) is contacted to the tumor to thereby deliver the molecules to the tumor cells. In one embodiment, the tumor cells are subjected to a second event to thereby activate the therapeutic molecule.

The term “tumor” refers to the tissue mass or tissue type or cell type that is undergoing uncontrolled proliferation. A tumor can be benign or malignant. A benign tumor is characterized as not undergoing metastasis. A malignant cell is a cancer cell and can undergo metastasis. Tumors on which the method can be performed include, without limitation, adenoma, angio-sarcoma, astrocytoma, epithelial carcinoma, germinoma, glioblastoma, glioma, hamartoma, hemangioendothelioma, hemangiosarcoma, hematoma, hepato-blastoma, leukemia, lymphoma, medulloblastoma, melanoma, neuroblastoma, osteosarcoma, retinoblastoma, rhabdomyosarcoma, sarcoma, and teratoma. The tumor can be chosen from acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangio-carcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic, papillary serous adeno-carcinoma, pineal cell, pituitary tumors, plasmacytoma, pseudo-sarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor.

### Treatment of Disease

Another aspect of the invention relates to a method of treating a subject for a disease by administering a therapeutic molecule to the subject in the form of a therapeutic microvesicle preparation. In one embodiment the disease is a cell proliferative disorder. In one embodiment, the cell proliferative disorder is a benign tumor. In one embodiment, the cell proliferative disorder is cancer.

A tumor or a cancer in a subject can be treated by administering a therapeutically effective amount of a therapeutic microvesicle preparation containing a conditionally lethal molecule as the therapeutic molecule. In addition, the subject may undergo a second event that activates the conditionally lethal molecule. One such conditionally lethal protein is cytosine deaminase (CD) fused in-frame with uracil phosphoribosyltransferase (UPRT). The microvesicle preparation containing a mRNA encoding cytosine deaminase (CD) fused in-frame with uracil phosphoribosyltransferase (UPRT), and/or the cytosine deaminase (CD) protein fused in-frame with uracil phosphoribosyltransferase (UPRT), is administered to the subject to thereby contact tumor or cancer cells in the subject (e.g., by injection into the tumor). In addition, a therapeutically effective amount 5-FC is administered to the subject to thereby contact the tumor or cancer cells (e.g., by localized administration or targeting to the tumor or cancer cells).

Diseases which involve a natural deficiency in a native molecule can also be treated by the methods discussed herein by delivery of the native molecule to the deficient cells through administration of a therapeutic microvesicle discussed herein.

### Pharmaceutical Compositions

Pharmaceutical compositions and formulations for specified modes of administration, described herein are also encompassed by the present invention. In one embodiment, the microvesicle preparation described herein is an active ingredient in a composition comprising a pharmaceutically acceptable carrier. Such a composition is referred to herein as a pharmaceutical composition. A “pharmaceutically acceptable carrier” means any pharmaceutically acceptable means to mix and/or deliver the targeted delivery composition to a subject. The term “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the composition and is compatible with administration to a subject, for example a human. Such compositions can be specifically formulated for administration via one or more of a number of routes, such as the routes of administration described herein. Supplementary active ingredients also can be incorporated into the compositions. When an agent, formulation or pharmaceutical composition described herein, is administered to a subject, preferably, a therapeutically effective amount is administered. As used herein, the term “therapeutically effective amount” refers to an amount that results in an improvement or remediation of the condition.

### Administration

Administration of the pharmaceutical composition to a subject is by means which the therapeutic microvesicles contained therein will contact the target cell. The specific route will depend upon certain variables such as the target cell, and can be determined by the skilled practitioner. Suitable methods of administering a composition comprising a recombinant nucleic acid molecule of the present invention to a patient include any route of in vivo administration that is suitable for delivering a recombinant nucleic acid molecule or protein to a patient. The preferred routes of administration will be apparent to those of skill in the art, depending on the type of therapeutic molecule used, the target cell population, and the disease or condition experienced by the subject. Preferred methods of in vivo administration include, but are not limited to, intravenous administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracerebral, nasal, oral, pulmonary administration, impregnation of a catheter, and direct injection into a tissue. In an embodiment where the target cells are in or near a tumor, a preferred route of administration is by direct injection into the tumor or tissue surrounding the tumor. For example, when the tumor is a breast tumor, the preferred methods of administration include impregnation of a catheter, and direct injection into the tumor.

Intravenous, intraperitoneal, and intramuscular administrations can be performed using methods standard in the art. Aerosol (inhalation) delivery can also be performed using methods standard in the art (see, for example, Stribling et al., Proc. Natl. Acad. Sci. USA 189:11277-11281, 1992, which is incorporated herein by reference in its entirety). Oral delivery can be performed by complexing a therapeutic composition of the present invention to a carrier capable of withstanding degradation by digestive enzymes in the gut of an animal. Examples of such carriers, include plastic capsules or tablets, such as those known in the art.

One method of local administration is by direct injection. Direct injection techniques are particularly useful for administering a recombinant nucleic acid molecule to a cell or tissue that is accessible by surgery, and particularly, on or near the surface of the body. Administration of a composition locally within the area of a target cell refers to injecting the composition centimeters and preferably, millimeters from the target cell or tissue.

### Dosage and Treatment Regimen

The appropriate dosage and treatment regimen for the methods of treatment described herein will vary with respect to the particular disease being treated, the molecules being delivered, and the specific condition of the subject. The skilled practitioner is to determine the amounts and frequency of administration on a case by case basis. In one embodiment, the administration is over a period of time until the desired effect (e.g., reduction in symptoms is achieved). In one embodiment, administration is 1, 2, 3, 4, 5, 6, or 7 times per week. In one embodiment, administration is over a period of 1, 2, 3, 4, 5, 6, 7, 8, 9, or 10 weeks. In one embodiment, administration is over a period of 2, 3, 4, 5, 6 or more months. In one embodiment, treatment is resumed following a period of remission.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means ±1%.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention may be as defined in any one of the following numbered paragraphs.
1. A method of producing therapeutic microvesicles comprising the steps:
   a) isolating donor cells from a subject;
   b) genetically modifying the donor cells to express a therapeutic nucleic acid and/or protein; and
   c) isolating microvesicles (extracellular vesicles) produced by the donor cells; to thereby produce therapeutic microvesicles.
2. The method of paragraph 1, wherein the cells are genetically modified by transduction with an expression vector encoding the therapeutic nucleic acid and/or protein in expressible form.
3. The method of any one of paragraphs 1-2, wherein the therapeutic nucleic acid is selected from the group consisting of mRNA, shRNA, miRNAs, ncRNA, and aptamer.
4. The method of paragraph 3 wherein the mRNA encodes a therapeutic protein.
5. The method of any one of paragraphs 3-4, wherein the mRNA molecule encodes a protein that activates a prodrug.
6. The method of paragraph 5, wherein the protein is cytosine deaminase fused in frame with uracil phosphoribosyltransferase and the prodrug is 5-fluorocytosine (5-FC).
7. The method of any one of paragraphs 1-6, wherein the therapeutic protein is an enzyme.
8. The method of any one of paragraphs 1-6, wherein the therapeutic protein is selected from the group consisting of Caspase-3, Caspase 7, Caspase-9, Caspase-8, Bax, Bid, Bad, Bak, BCL2L11, p53, PUMA, Diablo/SMAC, and S-TRAIL.
9. The method of any one of paragraphs 1-8, wherein the therapeutic protein activates a prodrug.
10. The method of any one of paragraphs 1-9, wherein the therapeutic protein is naturally deficient in a disease.
11. The method of paragraph 1, wherein the therapeutic protein is cytosine deaminase fused in frame with uracil phosphoribosyltransferase, and the prodrug is 5-FC.
12. The method of any one of paragraphs 1-11, wherein step b) results in overexpression of the therapeutic nucleic acid and/or protein in the donor cells.
13. The method of any one of paragraphs 1-12, wherein the donor cells are selected from the group consisting of epithelial cells, skin fibroblasts, mast cells, T lymphocytes, B lymphocytes and dendritic cells.
14. A therapeutic microvesicle preparation produced by the method of any one of paragraphs 1-13.
15. A method of delivering a therapeutic molecule to a subject comprising, administering an effective amount of a therapeutic microvesicle preparation of paragraph 14 to the subject.
16. The method of paragraph 15, wherein administering is local or systemic.
17. The method of paragraph 16, wherein local administering is by injection.
18. The method of paragraph 17, wherein injection is into an organ or a tumor.
19. A method of delivering a therapeutic molecule to a cell comprising, contacting the cell with an effective amount of a therapeutic microvesicle preparation of paragraph 14.
20. The method of paragraph 19, wherein the cell is in vivo.
21. The method of any one of paragraphs 19-20, wherein the cell is within an organ or tumor.
22. The method of any one of paragraphs 15-21, wherein the therapeutic microvesicle preparation is produced ex vivo from donor cells of a subject.
23. A method of treating a subject for a tumor comprising,
   a) administering a therapeutically effective amount of a therapeutic microvesicle preparation containing a therapeutic molecule selected from the group consisting of a mRNA encoding cytosine deaminase (CD) fused in-frame with uracil phosphoribosyltransferase (UPRT), cytosine deaminase (CD) fused in-frame with uracil phosphoribosyltransferase (UPRT), or a combination thereof, to the subject to thereby contact the therapeutic microvesicle preparation to a tumor in the subject; and
   b) administering a therapeutically effective amount 5-FC to the subject to thereby contact the tumor.
24. The method of paragraph 23, wherein administering step a) is by injection into the tumor.
25. The method of paragraph 23, wherein administering step b) is systemic.
26. The method of any one of paragraphs 23-25, wherein the therapeutic microvesicle preparation was prepared from donor cells isolated from the subject.
27. The method of paragraph 26, wherein the donor cells are selected from the group consisting of epithelial cells, skin fibroblasts, mast cells, T lymphocytes, B lymphocytes and dendritic cells.

The invention is further illustrated by the following examples, which should not be construed as further limiting.

### EXAMPLES

Based on the capacity of MVs to transfer cargo in nature, in the present study MVs were evaluated for whether they can serve as a novel cell-derived gene delivery vehicle carrying therapeutic mRNA/protein for cancer treatment. To test this hypothesis, cells were generated which stably expressed the suicide therapeutic mRNA/protein for cytosine deaminase (CD) fused in-frame with uracil phosphoribosyltransferase (UPRT), previously shown to be a potent prodrug activating combination.²⁸ MVs were harvested from these cells and used to treat schwannoma tumors. CD converts 5-fluorocytosine (5-FC) to 5-fluorouracil (5-FU), which is especially toxic to cells expressing UPRT due to its conversion to 5-fluoro-deoxyuridine monophosphate (5-FdUMP), an irreversible inhibitor of thymidine synthetase, thereby restricting the production of dTMP and downstream phosphorylated products. Depletion of dTTP results in inhibition of DNA synthesis and causes cells to go under apoptosis.^{29,30} With over-expression of the suicide gene in donor cells, high amounts of CD-UPRT message and protein were incorporated into MVs. These MVs were capable of transferring this therapeutic mRNA/protein to target tumor cells thereby achieving high level expression of functional protein in these recipient cells. Two different in vivo experiments resulted in significant inhibition of schwannoma tumor growth when CD-UPRT carrying MVs were injected into tumors in combination with systemic delivery of the prodrug, 5-FC.

### Results

### Use of Genetically Engineered MVs as a Novel Gene Delivery Tool

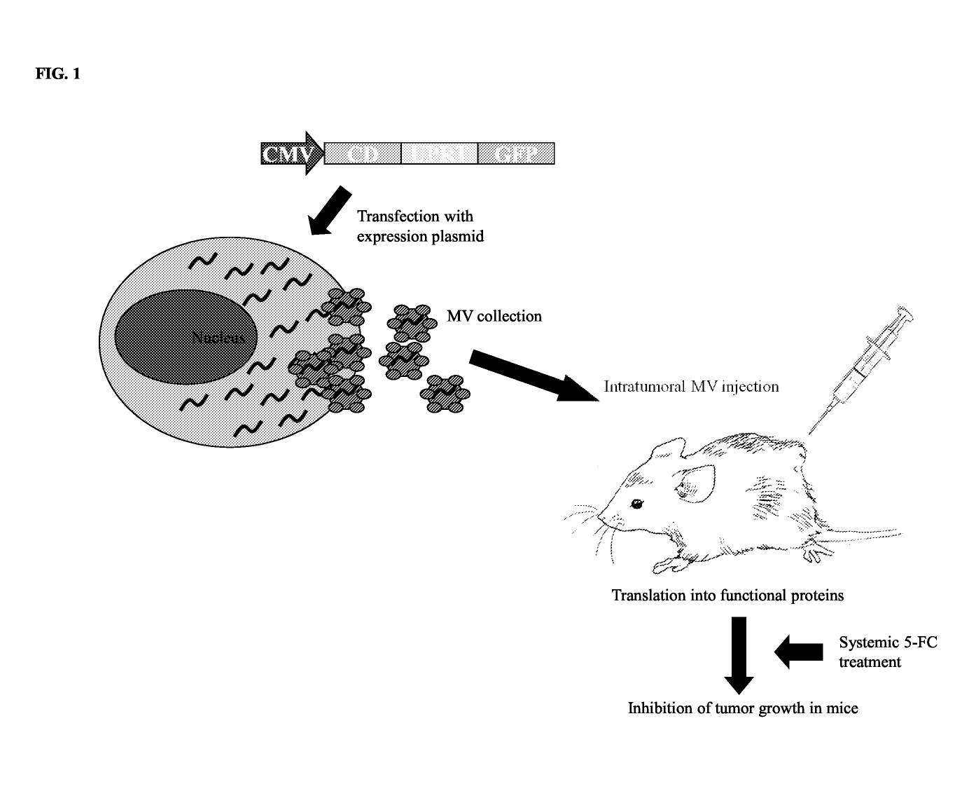

The general plan of action was to transduce donor cells with an expression cassette for a therapeutic gene, in this case CD-UPRT-EGFP (enhanced green fluorescent protein) under a strong promoter (the cytomegalovirus (CMV) promoter) and to allow time for high level expression of the cassette (72 hours). Then MVs were isolated from the conditioned medium by differential centrifugation, ultracentrifugation and filtration. These MVs were evaluated for enrichment of CD-UPRT-EGFP mRNA and protein (see below) and injected into tumors, followed, in this case, by systemic prodrug administration (5-FC) which should trigger apoptosis of tumor cells and regression of tumors. A schematic overview of the experiments performed in this study is illustrated in FIG. 1.

CD-UPRT-EGFP mRNA and Protein are Enriched in MVs

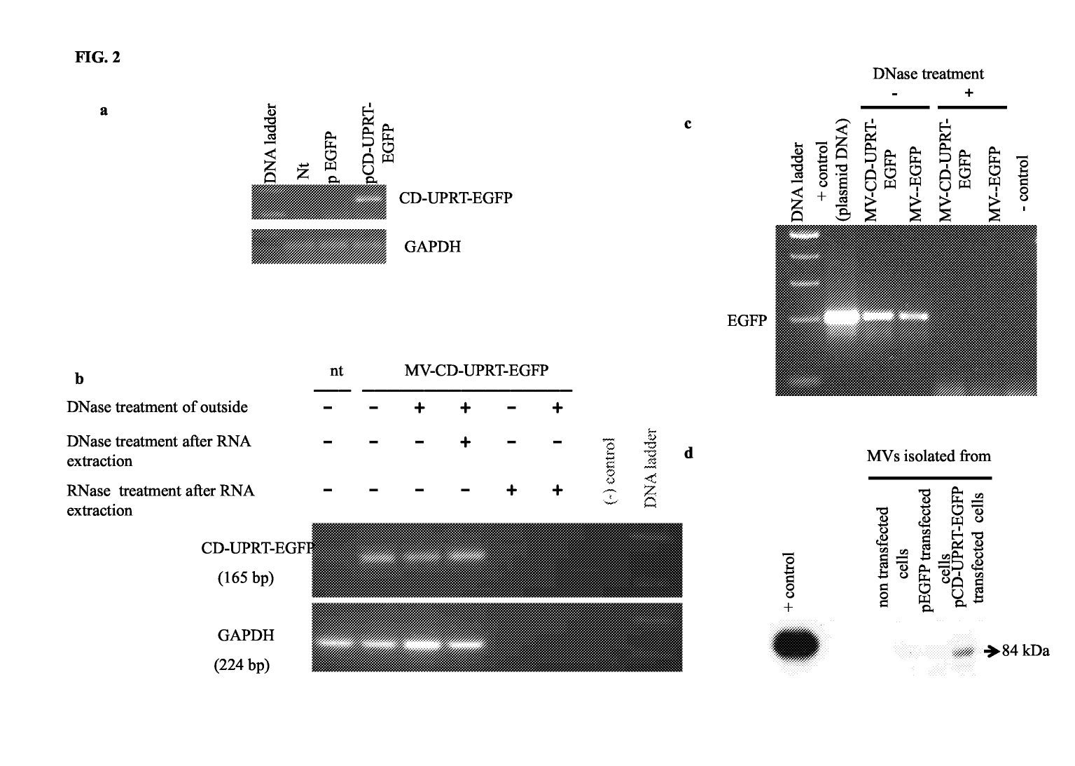

To test whether the CD-UPRT-EGFP mRNAs are enriched in MVs, HEK-293T were first transfected with either with pCD-UPRT-EGFP or pEGFP, and 3 days after transfection MVs were collected from medium and treated with DNaseI to remove any residual plasmid DNA bound to the surface of the MVs. Then RNA was isolated from MVs and qRT-PCRs were performed for CD-UPRT-EGFP and GAPDH mRNAs and end PCR products were loaded onto agarose gel. As shown in FIG. 2a, the CD-UPRT-EGFP mRNA was detected only in MVs isolated from pCD-UPRT-EGFP transfected cells, compared to control pEGFP or non-transfected cells (FIG. 2a). Moreover, to investigate the origin of the qRT-PCR signal, the following treatments were performed: with and without DNaseI on intact MVs and after release of MV contents, and RNase treatment after RNA isolation from MVs. As shown in FIG. 2b, DNaseI treatment on the outside of MVs did not prevent amplification of the RT-PCR product, whereas after RNase treatment of the contents of MVs, no signal was observed (FIG. 2b). In order to determine whether DNaseI treatment of MVs was functional, qPCR was performed directly (without RT) on the contents of isolated MVs and no PCR product was detected, supporting the conclusion that the PCR amplicons observed in the experimental conditions came entirely from mRNAs within MVs and not from any plasmid DNA contamination during MVs isolation (FIG. 2c). Since MVs can carry proteins as well as RNAs, whether CD-UPRT-EGFP protein was also incorporated into MVs was also analyzed. Western blots were performed directly on isolated MVs lysates using anti-CD antibody revealing that MVs from CD-UPRT-EGFP transfected cells also contained CD-UPRT-EGFP protein (FIG. 2d).

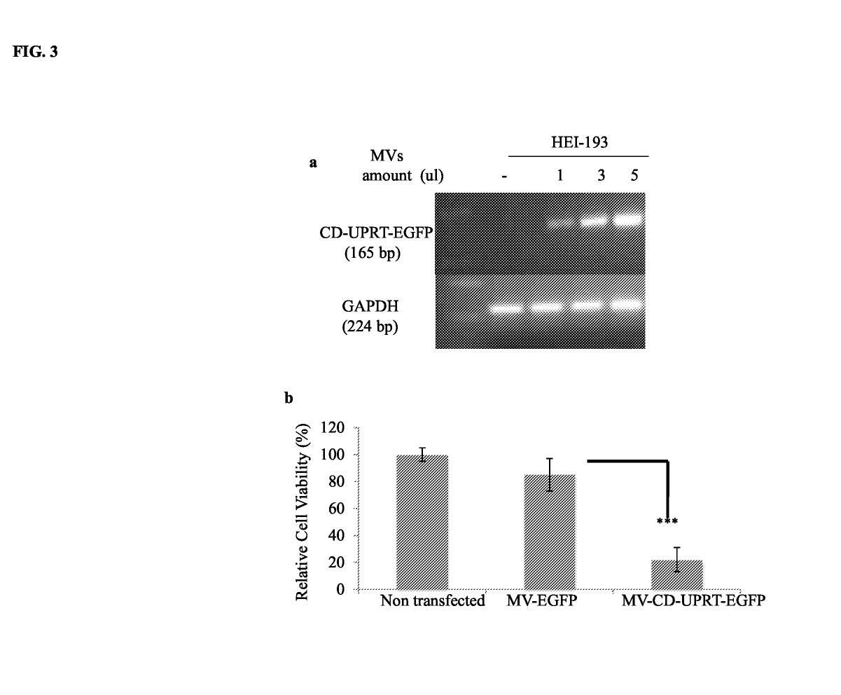

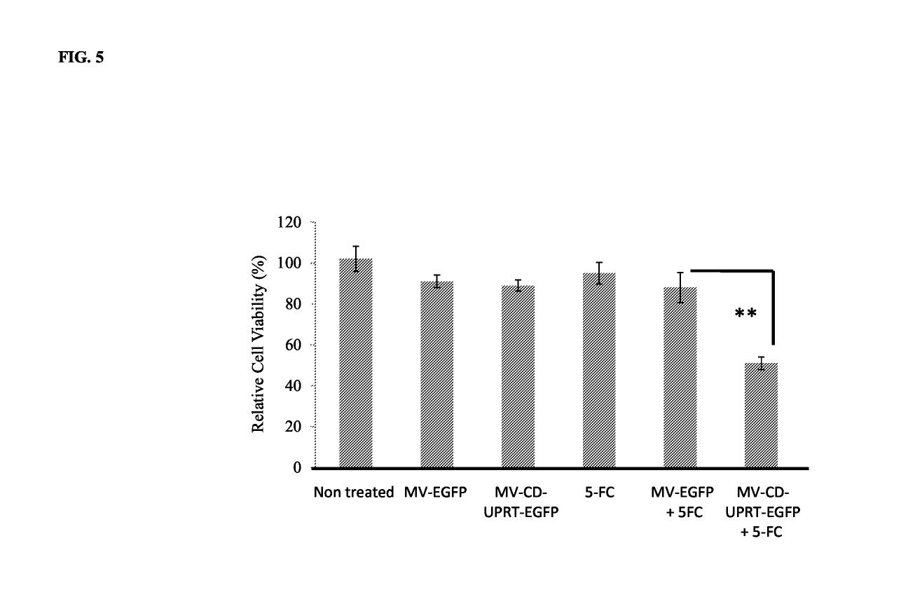

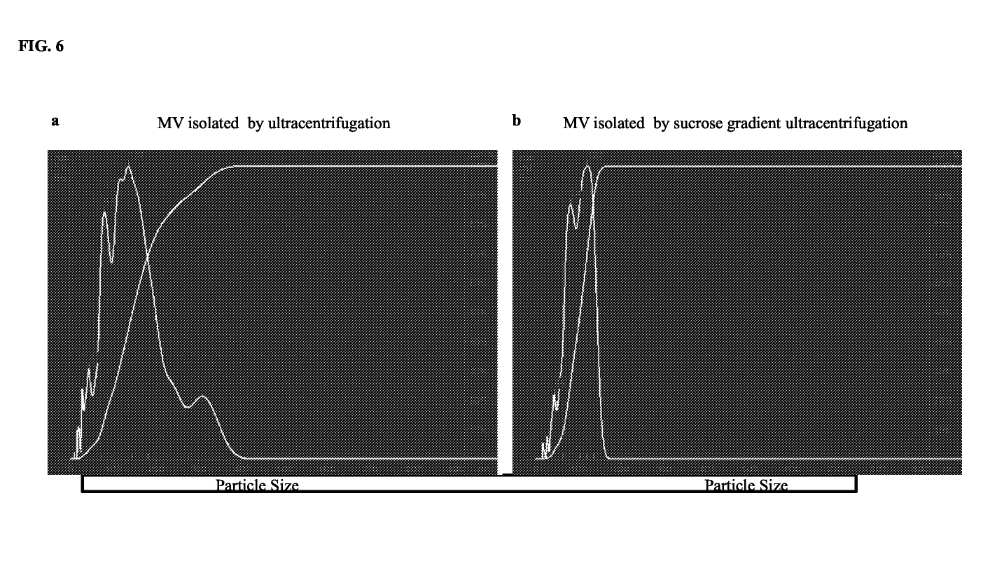

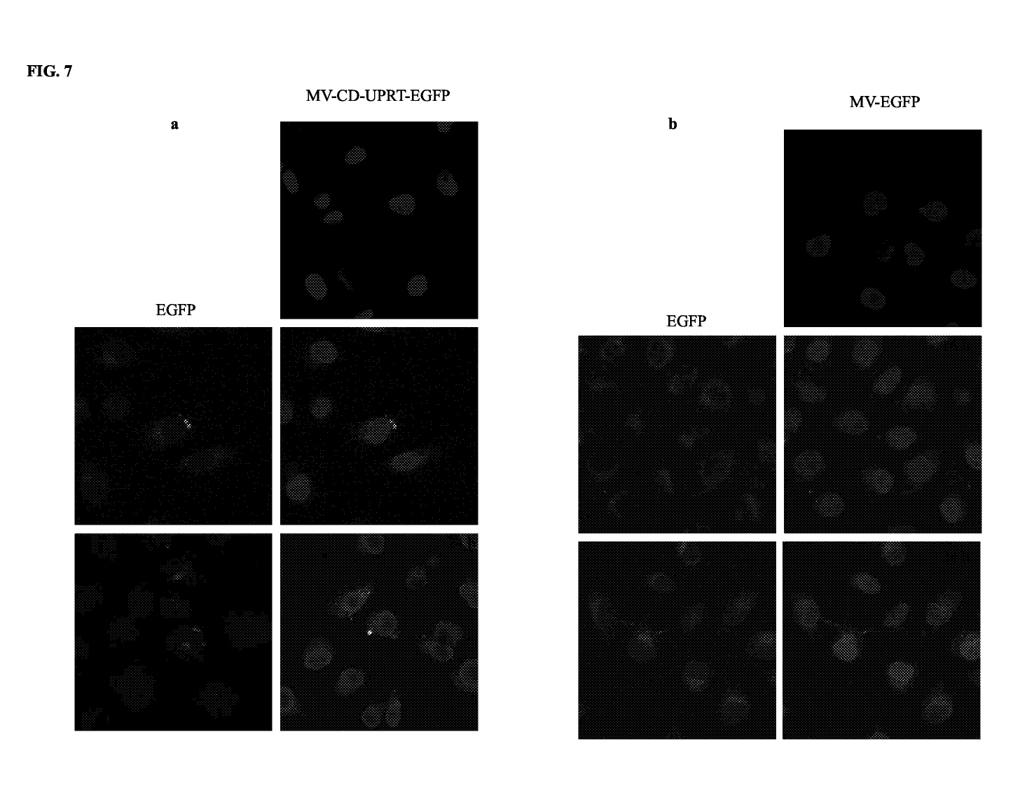

Whether MVs could mediate mRNA/protein delivery into recipient cells in culture was next examined. HEI-193 cells, human NF2 schwannoma cells immortalized with an oncogene³¹ were employed as recipient tumor cells based on a recently developed orthotopic schwannoma mouse model.^{32,33} Donor HEK-293T cells (4×10⁷) were transfected with pCD-UPRT-EGFP and 3 days later MVs were collected and concentrated as above in a 50 μl volume. HEI-193 cells (10⁵) were treated for two days with increasing volumes of MV concentrate—1, 3, and 5 μl corresponding to MVs released from 8×10⁵(1 μl), 2.4×10⁶ (3 μA) and 4×10⁶ (5 μl) cells, respectively. Then total RNA was isolated from recipient cells and RT-PCR was performed for the CD-UPRT-EGFP and GAPDH mRNAs. As shown in FIG. 3a, increasing amounts of the CD-UPRT-EGFP mRNA were observed in HEI-193 cells exposed to the higher numbers of MVs, supporting MV-mediated transfer of mRNA into the recipient cells. To test whether the delivery system was functional in culture, HEI-193 cells (10⁵) were treated with CD-UPRT-EGFP mRNA/protein enriched MVs (15 μl out of 50 μl MVs collected from 4×10⁷ donor cells) and 2 days later the prodrug, 5-FC was added to the medium followed by an MTT viability assay 24 hours later. HEI-193 cells treated with MVs carrying the CD-UPRT-EGFP mRNA/protein showed significant cell death (about 80%), compared to cells exposed to the control MVs carrying EGFP mRNA/protein after treatment with 5-FC (FIG. 3b). In order to evaluate any non-specific cell toxicity due to aggregated proteins which co-pelleted with MVs during MV isolation, MVs were also purified via sucrose gradient ultracentrifugation (fractions 3-7)³⁴ and the experiments performed in FIG. 3b were repeated. A pronounced and significant cell death (about 40%) in cells treated with these sucrose density isolated CD-UPRT-EGFP MVs was observed compared to cells exposed to control MVs (prepared in a similar manner) carrying EGFP mRNA/protein after treatment with 5-FC (FIG. 5). This supports the tumor toxicity of MVs, but does not exclude some portion being contributed by protein aggregates using centrifugal pelleting. The ultracentrifugation protocol used for MV isolation in FIG. 3b yielded MVs which were mostly 100-150 nm in diameter (mean 159 nm), but included an additional larger fraction 200-350 nm in diameter, whereas the sucrose gradient protocol yielded mostly MVs of a smaller size 50-60 nm in diameter (mean 104 nm; FIG. 6). Moreover, ultracentrifugation gave approximately 3-4 times more MVs than that sucrose gradient method from the same number of HEK-293T cells. The somewhat decreased cell death using sucrose density prepared MVs as compared to pelleted MVs observed in FIG. 3b may also be a consequence in the latter of larger MVs, known to be produced by tumorigenic cells²⁷ like HEK-293T cells carrying a greater amount per vesicle of the GFP-CD-UPRT mRNA/protein, as well as delivery of more MVs per cell. For treatment of tumor cells the number of MVs per cell was the same for MVs isolated by the two methods. Treatment of HEI-193 cells with MVs isolated with sucrose gradient method is also show in FIG. 7, which also provided evidence that larger MVs are lost during sucrose gradient preparation and produces relatively less MVs.

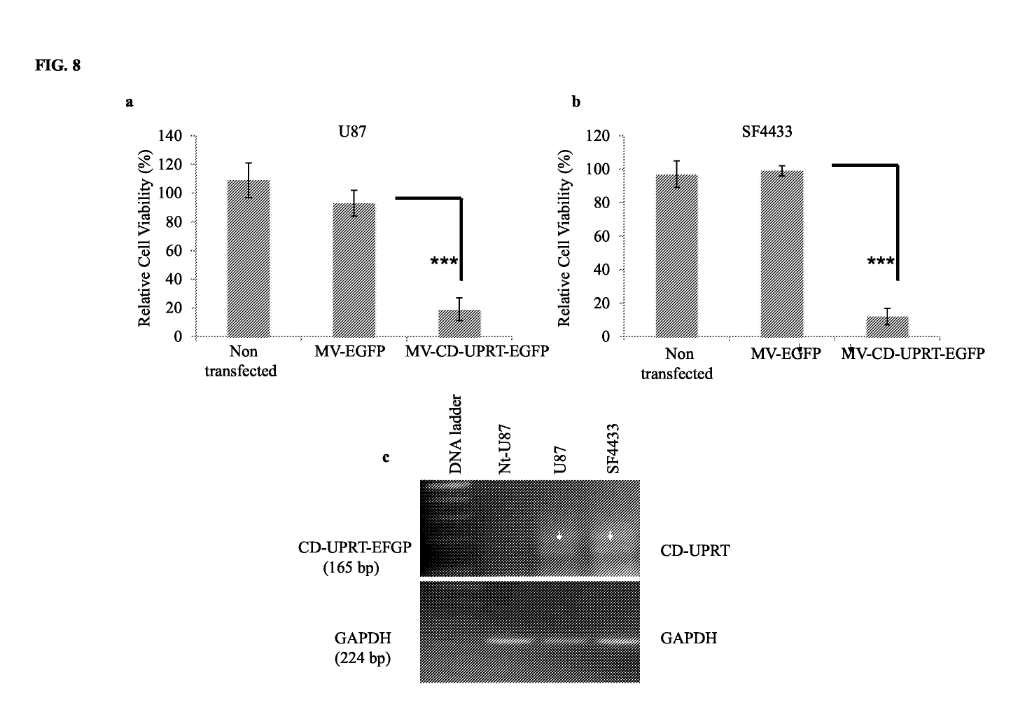

Treatment of other human cancer cell lines, glioblastoma (GBM) U87 and meningioma SF443 with the same concentration of CD-UPRT-EGFP-loaded MVs also resulted in significant cell death after prodrug administration in culture (FIG. 8a,b). Transfer of the CD-UPRT-EGFP mRNAs in those cells via MVs was confirmed with RT-PCR (FIG. 8c), together suggesting that this MV mediated suicide gene therapy approach is effective for a number of tumor cell types.

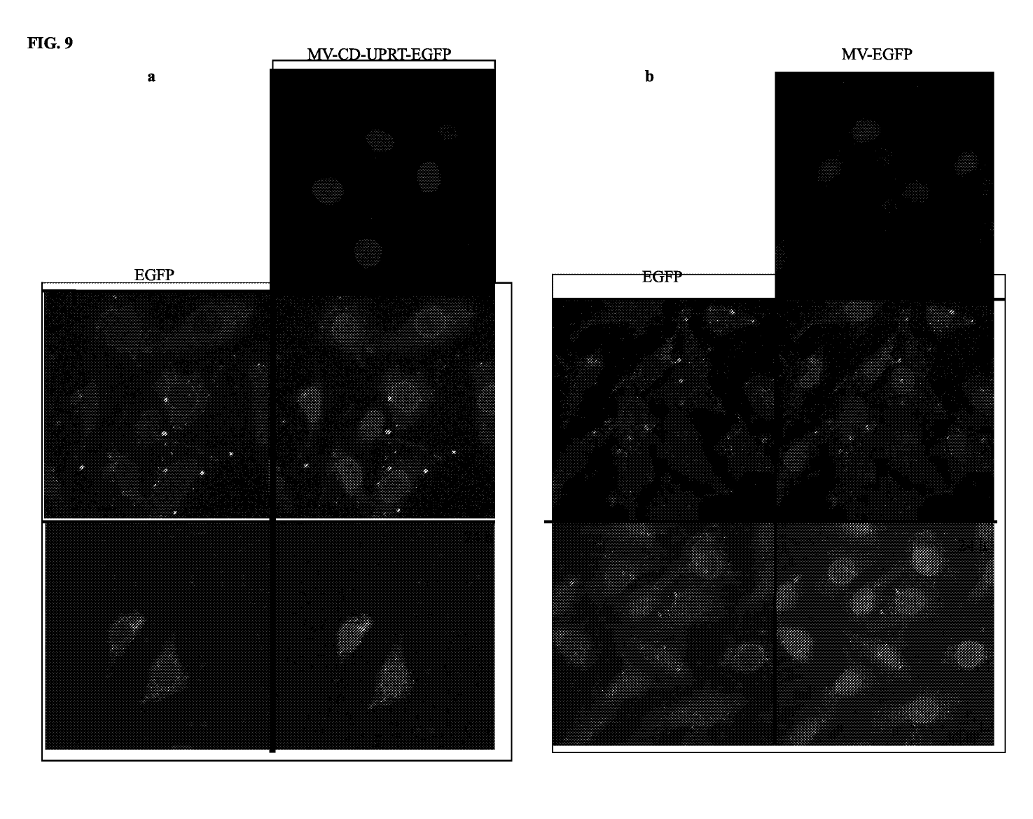

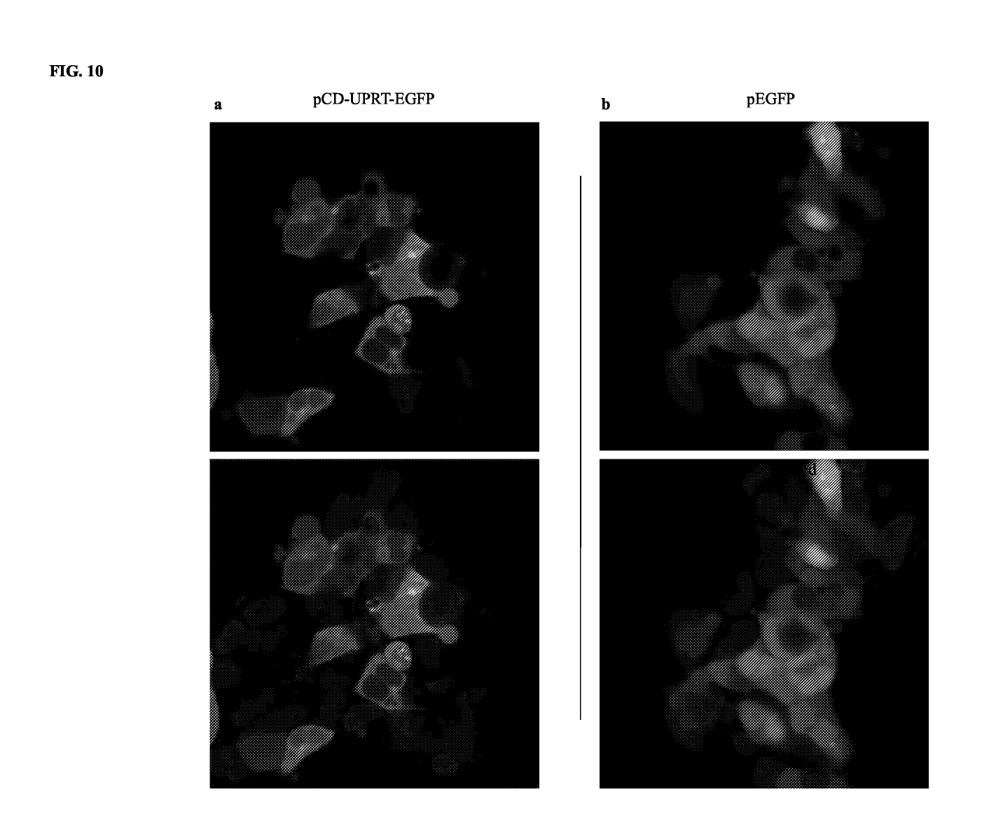

Under similar experimental conditions, HEI-193 cells were treated for 16 and 24 hours with MVs isolated from HEK-293T transfected with pCD-UPRT-EGFP or pEGFP and examined by fluorescence microscopy at indicated time points. In these experimental conditions, MVs were directly obtained from the medium of the transfected cells without any ultracentrifugation. Increased intracellular EGFP signal over time was observed in MV-CD-UPRT-EGFP and MV-EGFP treated cells indicating uptake of MVs by recipient cells with expression of EGFP continuing over at least 24 hours, apparently due at least in part to translation of the CD-UPRT-EGFP mRNA rather than just MV transfer of this protein (FIG. 9a,b). EGFP signal was observed prominently in the cytoplasm of the cells treated with MV-CD-UPRT-EGFP, whereas MV-EGFP treated cells exhibited a diffused EGFP signal both in nucleus and cytoplasm. Similar localization patterns of CD-UPRT-EGFP fusion protein and EGFP alone were also observed when they expressed from plasmid DNAs, pCD-UPRT-EGFP or pEGFP (FIG. 10).

Intratumoral Delivery of MVs Carrying CD-UPRT-EGFP mRNA/Protein Inhibits Schwannoma Tumor Growth In Vivo

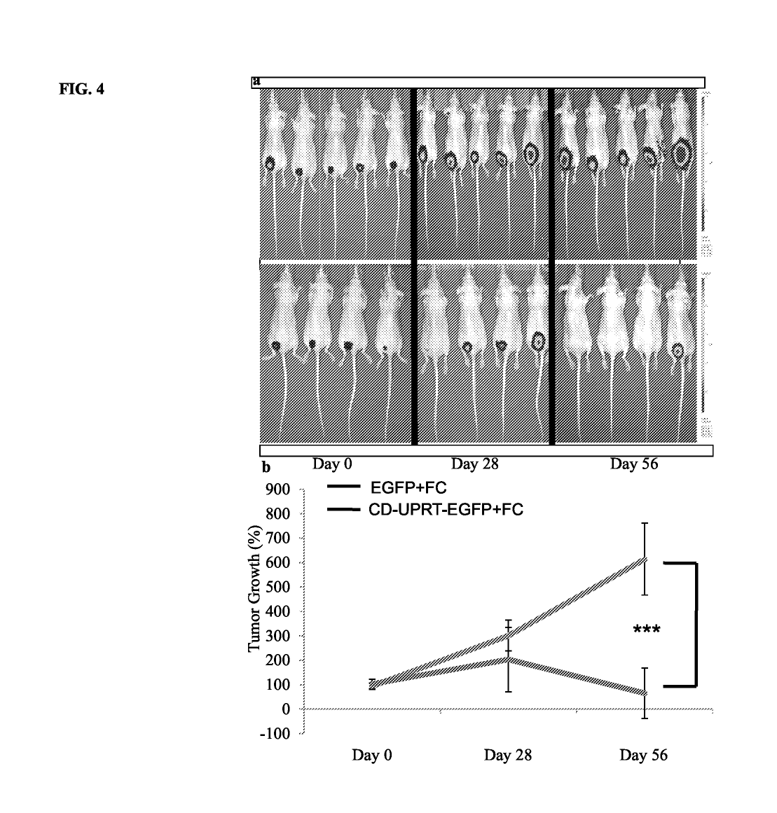

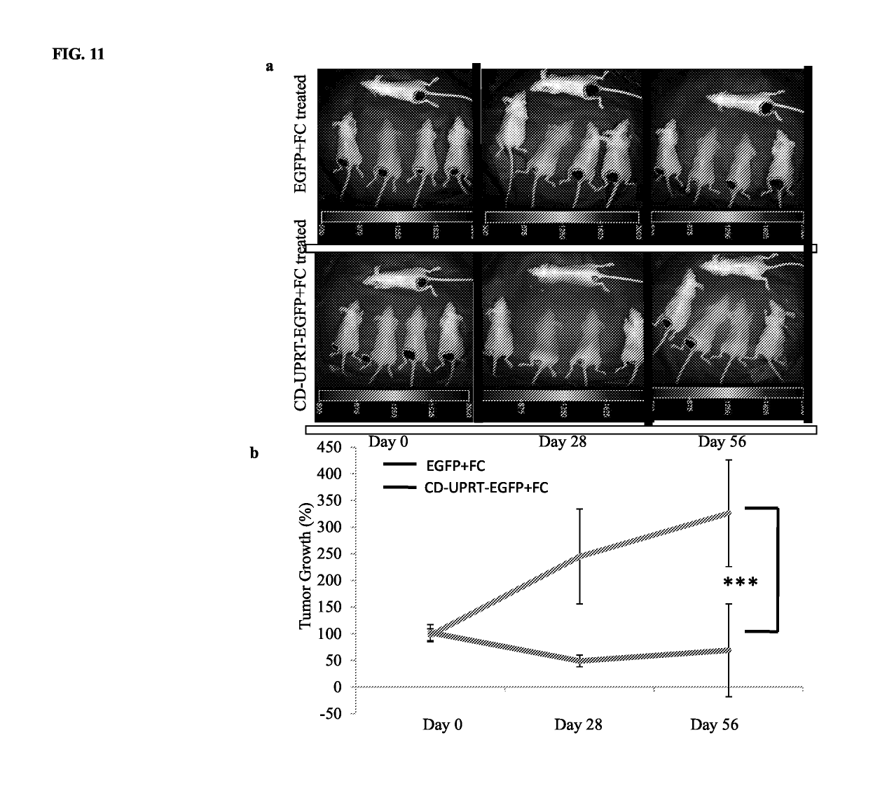

To test whether this therapeutic MV delivery system was functional in vivo, a recently developed orthotopic pre-established schwannoma tumor model was used.^{32,33} HEI-193 cells were stably transduced to express firefly luciferase (Fluc) and mCherry (mCh), yielding HEI-193FC cells.³⁵ In order to establish tumors, 3×10⁴ HEI-193FC cells in 1 μl of medium were implanted directly into the sciatic nerve of nude mice. Tumor development was monitored by in vivo bioluminescence imaging over three weeks and mice were re-grouped so that both groups harbored a range of similarly sized tumors based on photon counts. MVs carrying CD-UPRT-EGFP mRNAs/protein or EGFP mRNA/protein were prepared under similar experimental conditions and resuspended in 20 μl. One μl of the MVs were injected into each tumor once a week for two months, with fresh, loaded MVs prepared for each injection. Prodrug, 5-FC was administered intraperitoneally at daily intervals (12 mg/day) after the first MV injection. Two independent in vivo studies were conducted using a total of 10 mice in the control group (EGFP+5-FC treated) and 9 in the treatment group (CD-UPRT-EGFP+5-FC treated). Tumor growth was monitored by in vivo bioluminescence imaging at 28 day intervals. Bioluminescence images of tumors in these mice in 2 independent experiments are shown in FIG. 4a and FIG. 11a including quantification of the average photon counts. As shown in FIG. 4 and FIG. 11 in the treatment groups (CD-UPRT-EGFP+5-FC), tumor growth was completely inhibited in 6 of 9 mice, whereas all tumors in EGFP+5-FC control mice continued to grow. The lack of regression of the tumor in 3 of the treated mice is believed to be due to the difficulty in injecting directly into the tumor within the sciatic nerve. In control groups, in vivo imaging was terminated at day 56 because of excessive size of tumors. In the treatment group, in vivo imaging was carried out for an additional 2 weeks beyond that and none of the mice developed tumors.

### Materials and Methods

Plasmids.

Yeast CD—yeast UPRT ORF (pORF-FcyFur, Invitrogen, Grand Island, N.Y.) was fused to the N terminus of EGFP in pEGFP-N1 plasmid (Clontech, Mountainview, Calif.) under the CMV promoter and the plasmid construct is referred to as pCD-UPRT-EGFP in these studies. Forward 5′-GCTTCGAATTCATGGTCACAGGAGGCATGGCTTC (SEQ ID NO: 1) and reverse 5′-GACCGGTGGATCCACACAGTAGTATCTGTCCC (SEQ ID NO: 2) primers were used to amplify CD::UPRT by conventional PCR with Pfu Polymerase (Stratagene, Santa Clara, Calif.) in cloning steps.

Cells.

HEK-293T cells (from Dr. Maria Calos, Stanford University) were cultured in Dulbecco's Modified Eagle Medium (DMEM, Cellgro, Mediatech Inc., Manassas, Va.) containing 10% fetal bovine serum (FBS). Human schwannoma cell line HEI-193 established from a schwannoma tumor from a NF2 patient and immortalized with retroviral mediated HPV E6-E7 transduction (from Dr. David Lim, House Ear Institute, Los Angeles, Calif.) was cultured, as described.³¹ These cells were transduced with a lentivirus vector expressing Fluc and mCh, termed HEI-193FC cells, as described.³⁵ Meningioma cells, SF443 (from Dr. Anita Lal, University of California, San Francisco) were cultured, as described previously.^{50,51} U87 cells (ATCC) were cultured in DMEM containing 10% FBS. All cells were grown in the presence of 100 IU/ml penicillin and 100 μg/ml streptomycin and incubated at 37° C. in a 5% CO₂ atmosphere. Cells were determined to be mycoplasma negative by testing with a mycoplasma detection kit (MycoAlert® Mycoplasma Detection Assay: Lonza, Rockland, Me.).

MVs Isolation.

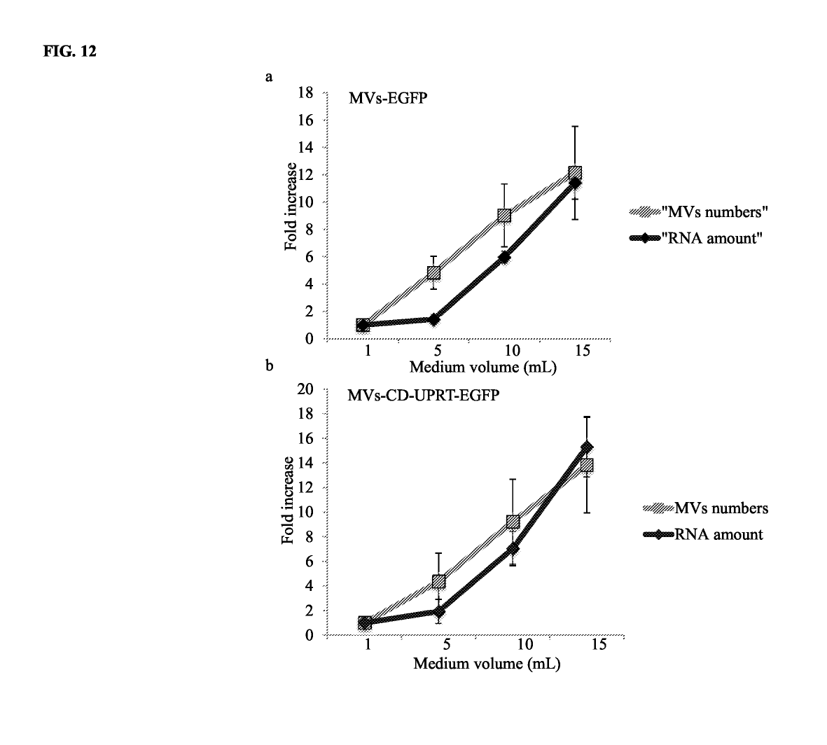

HEK-293T cells (maintained within 10 passages) were transfected either with pEGFP-N1 or pCD-UPRT-EGFP using Lipofectamine 2000 (Invitrogen, Grand Island, N.Y.), according to manufacturer's protocol. Five hours later, transfection media was replaced with media containing 5% MV-free FBS.²² Three days after transfection, MVs were harvested, as described.²² Briefly, culture medium from 2×150 mm plates containing 2×10⁷ cells per plate was first centrifuged at 300 g for 15 minutes to separate cells from medium, then at 16,000 g for 30 minutes to precipitate cellular debris. The supernatants were filtered through 0.22 μm filters (Millex, Billerica, Mass.) and then a final ultracentrifugation was performed at 110,000 g for 80 minutes using Beckman Quick seal tubes and a 70Ti rotor (Beckman Coulter, Fullerton, Calif.). Pelleted MVs were eluted either in 50 μl (for in vitro experiments) or 20 μl (for in vivo studies) in a mixture containing 1×PBS, RNase inhibitor (2 μl, 10U-RNAse-OUT—Invitrogen) and rDNase I (1 μl, 2U-DNA-free—Ambion). One μl MVs were delivered into each tumor once a week for two months, with fresh MVs prepared for each injection. Every 2 weeks, new, early passage cultures of the cells were generated to isolate MVs to make sure that there were no extended passage differences between the cells from which the MVs were isolated. MV yields were determined by measuring total RNA content using a NanoDrop (Wilmington, Del.) which correlated directly with MV number as assessed using a Nanosight, NS500 (FIG. 12).

For in vivo studies, MVs were prepared in the same manner and similar amounts of MVs were used in both groups as assessed by total RNA content: 189 ng±21 in 20 μL for CD-UPRT-EGFP MVs and 201 ng±38 in 20 μl for the control EGFP MVs. In some cases MVs were treated with 1 μl DNaseI (2U, Ambion, Grand Island, N.Y.) in 50 μl of total reaction for 30 minutes at 37° C. to remove DNA bound to the surface. In order to make sure that the DNase treatment protocol was functional, qPCR reactions were performed in the absence of RT directly from the MVs content and found no PCR amplification suggesting that the treatment protocol completely removed any plasmid DNA purified during MVs isolation.

Sucrose Gradient Ultracentrifugation.

Sucrose gradient ultracentrifugation was performed, as described previously.³⁴ Briefly, MVs were layered onto a sucrose density gradient (8, 30, 45, 60% layers) and centrifuged 38 minutes at 50,000 r.p.m. in SW40Ti swinging bucket rotor (Beckman Coulter) in a Beckman Optima ultracentrifuge with deceleration set to slow. Fractions 3-7 (density: Fraction 3 contains: half 8%, half 30%, Fraction 4 and 5 contains 30% only Fraction 6 and 7 contains 45% only) were collected, diluted in PBS and MVs pelleted at 100,000 g for 75 minutes in the S50A rotor using a Sorvall MX-120 microcentrifuge (Thermo Fisher Scientific, Agawam, Mass.) and used for western blot analysis for MV-associated proteins.

Total RNA isolation, reverse transcription and quantitative PCR. RNA was isolated from MVs using the miRvana kit (Ambion), according to manufacturer's protocol. Reverse transcription reaction was performed with 150 ng of MV RNA using Omniscript (Qiagen, Valencia, Calif.). Relative mRNA amounts were quantified with Applied Biosystems 7000 series quantitative PCR using SYBR Green (Applied Biosystems, Grand Island, N.Y.). Forward 5′-CACAACATGAGGTTCCAGAA (SEQ ID NO: 3) and reverse 5′-GAAGTTGACATTCTCTCCCA (SEQ ID NO: 4) primers were used to detect CD-UPRT-GFP message and forward 5′-GAAGGTGAAGGTCGGAGT (SEQ ID NO: 5) and reverse 5′-GAAGATGGTGATGGGATTTC (SEQ ID NO: 6) primers for GAPDH mRNA. Threshold cycles (CT) were analyzed using the Delta-ct formula and normalized to GAPDH mRNA levels. The qRT-PCR analysis was based on threshold cycles of the non-transfected cells derived MVs (nt-MVs) and the CD-UPRT-GFP transfected cell derived MVs (CD-MVs). The fold enrichment was calculated by comparing CT values. The highest CT value of qPCR (CT=30), indicating non-detectable levels of mRNA, was taken as our reference point in non-transfected (nt) and control plasmid, pEGFP, transfected cells and normalized to GAPDH CT values. Although the nt-MVs did not have the CD-UPRT-GFP message, the delta-CT value of the nt-MV samples were considered as 1 (CT=30) in order to determine the minimal level of enrichment of CD message in CD-UPRT-MVs.

Western Blot Analysis.

MVs were collected as described above and total protein (40 μg/lane) was resolved by electrophoresis in SDS-8% polyacrylamide gels and blotted onto nitrocellulose membranes, as described previously.⁵¹ The primary antibodies used were CD (Cell Signaling Technology, #4012, Danvers, Mass.) at 1:1000 dilution, and β-actin (Sigma, #A5441, St. Louis, Mo.) at 1:1000 dilution. Goat-anti-mouse IgG HRP conjugated (DAKO, Glostrup, Denmark, 1:5000, Cat# PO447) was used as a secondary antibody.

Cell Viability Assay.

HEI-193 cells were seeded (5000 per well) into 96-well plates one day before MV introduction. MVs were harvested from HEK-293 cells and eluted in 50 μl of cocktail mix (see above) and 15 μl was added into the medium in each well. Cells were incubated with MVs for 2 days before prodrug treatment and then treated with 250 μg/ml 5-FC (Invitrogen cat # sud-5fc). Three days later, MTT (Invitrogen) assays were performed, according to manufacturer's protocol to quantify cell viability.

Schwannoma Tumor Development and Bioluminescence Imaging.

Schwannoma tumors were developed as described previously.^{32,33} Briefly, HEI-193FC cells were trypsinized and rinsed, and then 3×10⁴ cells in 1 μl culture medium were injected directly into the sciatic nerve of athymic mice (nu/nu, 5-week-old females; Cox 7 breeding facility, MGH). In vivo bioluminescence imaging was performed, as described previously.^{32,33}

### Discussion

This study provides the first evidence for the potential therapeutic use of cell-derived MVs as novel and natural gene delivery vehicles for cancer treatment. In this strategy the “donor” cells were genetically engineered to express high levels of a conditionally therapeutic message and protein which are then incorporated into MVs derived from these cells. HEK-293T cells were first transfected with pCD-UPRT-EGFP plasmid and then allowed the CD-UPRT mRNA/protein to be incorporated into the MVs for 3 days followed by isolation and concentration of the MVs by ultracentrifugation. It was shown that both the CD-UPRT-EGFP message and protein were enriched in these MVs and were collectively functional. When recipient tumor cells were exposed to these loaded MVs and the prodrug, 5-FC it was converted to its active form leading to cell death. Furthermore, in in vivo studies, when pre-established mouse sciatic nerve schwannomas were injected intratumorally with CD-UPRT-mRNA/protein-bearing MVs and treated with 5-FC, there was marked inhibition of tumor growth and regression of tumor size in two independent studies. Without being bound by theory, it is thought that this therapeutic effect is mediated by both mRNA and protein delivery. Taken together, these data indicate that MVs can be used as a gene delivery tool to treat cancer.

Ideal therapeutic delivery vehicles should have a good packaging size and no immunogenic response in the host organism.¹ Due to the fact that MVs are stable enough to have a relatively long half life in tissues, small enough to diffuse throughout target tissues and large enough to carry sufficient amounts of genetic and protein material for different purposes, they may prove to be among the most potent biological gene/protein delivery vehicles.¹ Since therapeutic MVs can be derived from the host own cells placed in culture, they have the potential to evade the host's immune system, and with further modifications of membrane components, immunogenicity may be further decreased. For example, the host cells can be engineered to produce immunosuppressive ligands or cytokines which MVs derived from those engineered cells will also carry.³⁶ In addition, introduction of specific peptides, such as targeting moieties onto the MV membrane should serve to target MVs to specific cell populations³⁷ or tissues, such as the brain with access across the blood-brain barrier (BBB).³⁸ Further, these cell-derived “physiologic liposomes” can carry multiple components including miRNA, mRNA, non-coding regulatory RNAs, proteins and DNA (for review see²⁶). Interestingly, a recent study has revealed a zipcode-like 25 nt sequence in the 3′UTRs of many of the most enriched mRNAs in MVs derived from human primary GBM cells, which enhances mRNA incorporation into MVs, in part through interaction with miR-1289³⁸.

When compared to current viral gene delivery tools, MVs derived from an individual's cells should be recognized as “self” by the body, resulting in less immune response in the host organism to the delivery vehicle, as compared to virus vectors. Furthermore, some viral constructs also induce protein kinase R (PKR) stress signaling in host cells.⁴⁰ HSV also encodes proteins, e.g. UL41 (vhs) that block host cell protein translation,⁴¹ and these cellular and viral proteins can be toxic to cells in their own right and may interfere with action of therapeutic proteins⁴².

MVs can be taken up by endocytosis or fusion with the recipient cell plasma membrane as determined by membrane-bound protein interactions, after which genetic and protein material within the MVs are released into the cells. Although PKR signaling and many other ribonucleases recognize foreign bacterial or viral RNA/DNA molecules, MVs mRNA molecules should have the common eukaryotic signals, such as untranslated regions at the 5′ and 3′ terminals, capping at the 5′ terminal, and coding sequences, which are distinct from microbial mRNAs. In addition, the immune system should recognize MVs produced by normal cells as self due to immunologic memory as these MVs are present in all body fluids, including blood and urine.^{43,44} Several studies also support the functional nature of transferred miRNAs and mRNAs which modify the translational profile and phenotype of the recipient cells.^{23,22,45,38,46,47,48} A recent study also showed that MVs derived from genetically engineered dendritic cells can successfully transfer small-interfering-RNA (siRNA) molecules to mouse brain with consequent down-regulated translation of targeted mRNAs.³⁸ Taken together—less immunogenic response, abundant gene/protein transfer capacity and ample packaging size, cell-derived MVs have immense potential as therapeutic delivery vehicles for disease applications in the future. Two applications can be envisioned. In one strategy, cells from the affected individual would be placed in culture, genetically modified as needed for targeting and delivery of the therapeutic protein/RNA and then MVs isolated from cultures administered to the patient, as modeled in Alvarez-Erviti et al.³⁸ In the context of cancer therapy, it will be important that the therapeutic gene does not kill the donor cells, so, for example, it could be a prodrug activating enzyme, as in this study, or a protein such as a membrane-bound form of TRAIL which is not toxic to most normal cells, but kills tumor cells upon transfer.⁴⁹ In a second strategy, it should be possible to genetically modify cells in vivo so that they produce conditionally therapeutic MVs, such as the prodrug activating scheme used in this study to empower tumor cells so that they “become their own worst enemies”. The results presented here provide a proof of concept that MVs can serve as therapeutic delivery vehicles for cancer and other diseases.

### REFERENCES

1. Seow, Y and Wood, M J (2009). Biological Gene Delivery Vehicles: Beyond Viral Vectors. Mol Ther 17: 767-777.
2. Bae, K H, Chung, H J and Park, T G (2011). Nanomaterials for cancer therapy and imaging. Mol Cells 31: 295-302.
3. Takakura, Y, Nishikawa, M, Yamashita, F and Hashida, M (2001). Development of gene drug delivery systems based on pharmacokinetic studies. Eur J Pharm Sci 13: 71-76.
4. Zhang, J S, Liu, F and Huang, L (2005). Implications of pharmacokinetic behavior of lipoplex for its inflammatory toxicity. Adv Drug Deliv Rev 57: 689-698.
5. Coura Rdos, S and Nardi, N B (2007). The state of the art of adeno-associated virusbased vectors in gene therapy. Virol J 4: 99.
6. Hacein-Bey-Abina, S, Garrigue, A, Wang, G P, Soulier, J, Lim, A, Morillon, E et al. (2008). Insertional oncogenesis in 4 patients after retrovirus-mediated gene therapy of SCID-X1. J Clin Invest 118: 3132-3142.
7. Hartman, Z C, Appledorn, D M and Amalfitano, A (2008). Adenovirus vector induced innate immune responses: impact upon efficacy and toxicity in gene therapy and vaccine applications. Virus Res 132: 1-14.
8. Zaiss, A K and Muruve, D A (2005). Immune responses to adeno-associated virus vectors. Curr Gene Ther 5: 323-331.
9. Dobrovolskaia, M A, Aggarwal, P, Hall, J B and McNeil, S E (2008). Preclinical studies to understand nanoparticle interaction with the immune system and its potential effects on nanoparticle biodistribution. Mol Pharm 5: 487-495.
10. Hornung, V, Bauernfeind, F, Halle, A, Samstad, E O, Kono, H, Rock, K L et al. (2008). Silica crystals and aluminum salts activate the NALP3 inflammasome through phagosomal destabilization. Nat Immunol 9: 847-856.
11. van den Boom, J G, Schlee, M, Coch, C and Hartmann, G (2011). SiRNA delivery with exosome nanoparticles. Nat Biotechnol 29: 325-326.
12. Cocucci, E, Racchetti, G and Meldolesi, J (2009). Shedding microvesicles: artefacts no more. Trends Cell Biol 19: 43-51.
13. van Niel, G, Porto-Carreiro, I., Simoes, S and Raposo, G (2006). Exosomes: a common pathway for a specialized function. J Biochem (Tokyo) 140: 13-21.
14. Stoorvogel, W, Kleijmeer, M J, Geuze, H J and Raposo, G (2002). The biogenesis and functions of exosomes. Traffic 3: 321.
15. Johnstone, R M (2006). Exosomes biological significance: A concise review. Blood Cells Mol Dis 36: 315-321.
16. Keller, S, Sanderson, M P, Stoeck, A and Altevogt, P (2006). Exosomes: from biogenesis and secretion to biological function. Immunol Lett 107: 102-108.
17. Shen, B, Wu, N, Yang, J M and Gould, S J (2011). Protein targeting to exosomes/microvesicles by plasma membrane anchors. J Biol Chem 286: 14383-14395.
18. Raposo, G, Nijman, H W, Stoorvogel, W, Liejendekker, R, Harding, C V, Melief, C J et al. (1996). B lymphocytes secrete antigen-presenting vesicles. J Exp Med 183: 1161-1172.
19. Blanchard, N, Lankar, D, Faure, F, Regnault, A, Dumont, C, Raposo, G et al. (2002).
TCR activation of human T cells induces the production of exosomes bearing the TCR/CD3/complex. J Immunol 168: 3235-3241.
20. Zitvogel, L, Regnault, A, Lozier, A, Wolfers, J, Flament, C, Tenza, D et al. (1998). Eradication of established murine tumors using a novel cell-free vaccine: dendritic cell-derived exosomes. Nat Med 4: 594-600.
21. Wolfers, J, Lozier, A, Raposo, G, Regnault, A, Théry, C, Masurier, C et al. (2001). Tumor derived exosomes are a source of shared tumor rejection antigens for CTL cross-priming. Nat Med 7: 297-303.
22. Skog, J, Würdinger, T, van Rijn, S, Meijer, D, Gainche, L, Curry, W T J et al. (2008). Glioblastoma microvesicles transport RNA and protein that promote tumor growth and provide diagnostic biomarkers. Nat Cell Biol 10: 1470-1476.
23. Valadi, H, Ekstrom, K, Bossios, A, Sjostrand, M, Lee, J J and Lotvall, J O (2007). Exosome-mediated transfer of mRNAs and microRNAs is a novel mechanism of genetic exchange between cells. Nat Cell Biol 9: 654-659.
24. Ratajczak, J, Miekus, K, Kucia, M, Zhang, J, Reca, R, Dvorak, P et al. (2006). Embryonic stem cell-derived microvesicles reprogram hematopoietic progenitors: evidence for horizontal transfer of mRNA and protein delivery. Leukemia 20: 847-856.
25. Al-Nedawi, K, Meehan, B, Micallef, J, Lhotak, V, May, L, Guha, A et al. (2008). Intercellular transfer of the oncogenic receptor EGFRvIII by microvesicles derived from tumour cells. Nat Cell Biol 10: 619-624.
26. van der Vos, K E, Balaj, L, Skog, J and Breakefield, X O (2011). Brain tumor microvesicles: Insights into intercellular communication in the nervous system. Cell Mol Neurobiol 31: 949-959.
27. Balaj, L, Lessard, R, Dai, L, Cho, Y-J, Pomeroy, S L, Breakefield, X et al. (2011). Tumour microvesicles contain retrotransposon elements and amplified oncogene sequences. Nat Commun 2: 180.
28. Erbs, P, Regulier, E, Kintz, J, Leroy, P, Poitevin, Y, Exinger, F et al. (2000). In vivo cancer gene therapy by adenovirus-mediated transfer of a bifunctional yeast cytosine deaminase/uracil phosphoribosyltransferase fusion gene. Cancer Res 60: 3813-3822.
29. Huber, B E, Austin, E A, Richards, C A, Davis, S T and Good (1994). Metabolism of 5-fluorocytosine to 5-fluorouracil in human colorectal tumor cells transduced with the cytosine deaminase gene: significant antitumor effects when only a small percentage of tumor cells express cytosine deaminase. Proc Natl Acad Sci USA 91: 8302-8306.
30. Mullen, C A, Coale, M M, Lowe, R and Blaese, R M (1994). Tumors expressing the cytosine deaminase suicide gene can be eliminated in vivo with 5-fluorocytosine and induce protective immunity to wild type tumor. Cancer Res 54: 1503-1506.
31. Hung, G, Li, X, Faudoa, R, Xeu, Z, Kluwe, L, Rhim, J S et al. (2002). Establishment and characterization of a schwannoma cell line from a patient with neurofibromatosis 2. Int J Oncol 20: 475-482.
32. Saydam, O, Ozdener, G B, Senol, O, Mizrak, A, Prabhakar, S, Stemmer-Rachamimov, A O et al. (2011). A novel imaging-compatible sciatic nerve schwannoma model. J Neurosci Methods 195: 75-77.
33. Saydam, O, Senol, O, Würdinger, T, Mizrak, A, Ozdener, G B, Stemmer-Rachamimov, A O et al. (2011). miRNA-7 attenuation in schwannoma tumors stimulates growth by upregulating three oncogenic signaling pathways. Cancer Res 17: 852-861.
34. Maguire, C A, Balaj, L, Sivaraman, S, Crommentuijn, M H, Ericsson, M, Mincheva-Nilsson, L et al. (2012). Microvesicle-associated AAV vector as a novel gene delivery system. Mol Ther 20: 960-971.
35. Prabhakar, S, Brennan, G J, Messerli, S M, Mao, J, Sena-Esteves, M, Stemmer-Rachamimov, A et al. (2010). Imaging and therapy of experimental schwannomas using HSV amplicon vector-encoding apoptotic protein under Schwann cell promoter. Cancer Gene Ther 17: 266-274.
36. Kim, S H, Bianco, N, Menon, R, Lechman, E R, Shufesky, W J, Morelli, A E et al. (2006). Exosomes derived from genetically modified D C expressing FasL are anti-inflammatory and immunosuppressive. Mol Ther 13: 289-300.
37. Maguire, C A, Balaj, L, Sivaraman, S, Crommentuijn, M, Ericsson, M, Mincheva-Nilsson, L et al. (2012). Microvesicle-associated AAV vector as a novel gene delivery system. Mol Ther February 7: [Epub ahead of print].
38. Alvarez-Erviti, L, Seow, Y, Yin, H, Betts, C, Lakhal, S and Wood, M J (2011). Delivery of siRNA to the mouse brain by systemic injection of targeted exosomes. Nat Biotechnol 29: 341-345.
39. Bolukbasi, M F, Mizrak, A, Ozdener, B G, Madlener, S, Strobel, T, Skog, J et al. miR-1289 and “zipcode”-like sequence enrich mRNAs in microvesicles. Mol Ther-Nucleic Acids, in press.
40. Patel, C V, Handy, I, Goldsmith, T and Patel, R C (2000). PACT, a stress-modulated cellular activator of interferon-induced double-stranded RNA-activated protein kinase, PKR. J Biol Chem 275: 37993-37998.
41. Matis, J and Küdelová, M (2001). Early shutoff of host protein synthesis in cells infected with herpes simplex viruses. Acta Virol 45: 269-277.
42. García, M A, Meurs, E F and Esteban, M (2007). The dsRNA protein kinase PKR: virus and cell control. Biochimie 89: 799-811.
43. Caby, M P, Lankar, D, Vincendeau-Scherrer, C, Raposo, G and Bonnerot, C (2005). Exosomal-like vesicles are present in human blood plasma. Int Immunol 17: 879-887.
44. Gonzales, P, Pisitkun, T and Knepper, M A (2008). Urinary exosomes: is there a future? Nephrol Dial Transplant 23: 1799-1801.
45. Pegtel, D M, Cosmopoulos, K, Thorley-Lawson, D A van Eijndhoven, M A, Hopmans, E, Lindengey, J L et al. (2010). Functional delivery of viral miRNAs via exosomes. Proc Natl Acad Sci USA 107: 6328-6333.
46. Yuan, A, Farber, E L, Rapoport, A L, Tejada, D, Deniskin, R, Akhmedov, N B and Farber, D B (2009). Transfer of microRNAs by embryonic stem cell microvesicles. PLoS One 4: e4722.
47. Mittelbrunn, M, Gutiérrez-Vázquez, C, Villarroya-Beltri, C, González, S, Sánchez-Cabo, F, González, M. Á et al. (2011). Unidirectional transfer of microRNA-loaded exosomes from T cells to antigen-presenting cells. Nat Commun 2: 282.
48. Montecalvo, A, Larregina, A T, Shufesky, W J, Beer Stolz, D, Sullivan, M L, Karlsson, J M et al. (2012). Mechanism of transfer of functional microRNAs between mouse dendritic cells via exosomes. Blood 119: 756-766.
49. Shah, K, Bureau, E, Kim, D E, Yang, K, Tang, Y, Weissleder, R et al. (2005). Glioma therapy and real-time imaging of neural precursor cell migration and tumor regression. Ann Neurol 57: 34-41.
50. Cuevas, I C, Slocum, A L, Jun, P, Costello, J F, Bollen, A W, Riggins, G J et al. (2005). Meningioma transcript profiles reveal deregulated Notch signaling pathway. Cancer Res 65: 5070-5075.
51. Saydam, O, Shen, Y, Wiirdinger, T, Senol, O, Boke, E, James, M F et al. (2009). Downregulated microRNA-200a in meningiomas promotes tumor growth by reducing E-cadherin and activating the Wnt/beta-catenin signaling pathway. Mol Cell Biol 29: 5923-5940.

1. A method of producing therapeutic microvesicles comprising the steps:
a) isolating donor cells from a subject;
b) genetically modifying the donor cells to express a therapeutic nucleic acid and/or protein; and
c) isolating microvesicles (extracellular vesicles) produced by the donor cells;
to thereby produce therapeutic microvesicles.

2-27. (canceled)
